SEQUENCE LISTING (117 sequences)

SEQ ID NO: 1 - protein, 10526 aa, Bombyx mori
MRVKTFVILCCALQYVAYTNANINDFDEDYFGSDVTVQSSNTTDEIIRDASGAVIEEQITTKKMQRKNKNHGILGKNEKMIKTFVITTDSDGNESIVEEDVLMKTLSDGTVAQSYVAADAGAYSQSGPYVSNSGYSTHQGYTSDFSTSAAVGAGAGAGAAAGSGAGAGAGYGAASGAGAGAGAGAGAGYGTGAGAGAGAGYGAGAGAGAGAGYGAGAGAGAGAGYGAGAGAGAGAGYGAGAGAGAGAGYGAGAGAGAGAGYGAASGAGAGAGYGQGVGSGAASGAGAGAGAGSAAGSGAGAGAGTGAGAGYGAGAGAGAGAGYGAASGTGAGYGAGAGAGYGGASGAGAGAGAGAGAGAGAGYGTGAGYGAGAGAGAGAGAGAGYGAGAGAGYGAGYGVGAGAGYGAGYGAGAGSGAASGAGSGAGAGSGAGAGSGAGAGSGAGAGSGAGAGSGAGAGSGAGAGSGAGAGSGTGAGSGAGAGYGAGAGAGYGAGAGSGAASGAGAGSGAGAGSGAGAGSGAGAGSGAGAGSGAGAGYGAGAGAGYGAGAGAGYGAGAGVGYGAGAGSGAASGAGAGSGAGAGSGAGAGSGAGAGSGAGAGSGAGAGSGAGAGSGAGAGSGAGAGSGAGVGYGAGVGAGYGAGYGAGAGAGYGAGAGSGAASGAGAGAGAGAGTGSSGFGPYVANGGYSRSDGYEYAWSSDFGTGSGAGAGSGAGAGSGAGAGSGAGAGSGAGAGSGAGAGYGAGVGVGYGAGYGAGAGAGYGAGAGSGAASGAGAGSGAGAGSGAGAGSGAGAGSGAGAGSGAGAGSGAGAGSGAGAGSGAGAGSGAGAGSGAGVGSGAGAGSGAGAGVGYGAGAGVGYGAGAGSGAASGAGAGSGAGAGSGAGAGSGAGAGSGAGAGSGAGAGSGAGAGSGAGAGSGAGAGSGAGVGYGAGVGAGYGAGYGAGAGAGYGAGAGSGAASGAGAGSGAGAGSGAGAGSGAGAGSGAGAGSGAGAGSGAGAGSGAGAGSGAGAGSGAGAGSGAGAGSGAGAGYGAGAGAGYGAGYGAGAGAGYGAGAGSGAASGAGSGAGAGSGAGAGAGSGAGAGSGAGAGSGAGAGSGAGAGSGAGAGSGAGAGYGAGVGAGYGAGYGAGAGAGYGAGAGSGAASGAGAGSGAGAGSGAGAGSGAGAGSGAGAGSGAGAGSGAGAGSGAGVGYGAGYGAGAGAGYGAGAGSGAASGAGAGAGAGAGTGSSGFGPYVAHGGYSGYEYAWSSESDFGTGSGAGAGSGAGAGSGAGAGSGAGAGSGAGYGAGVGAGYGAGYGAGAGAGYGAGAGSGAGSGAGAGSGAGAGSGAGAGSGAGAGSGAGAGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGAGYGAGVGAGYGAGYGAGAGAGYGAGAGSGAGSGAGAGSGAGAGSGAGAGSGAGVGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGVGYGAGVGAGYGAGYGAGAGAGYGAGAGSGAASGAGAGAGAGAGTGSSGFGPYVANGGYSGYEYAWSSESDFGTGSGAGAGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGSGSGAGAGSGAGAGSGAGAGYGAGVGAGYGVGYGAGAGAGYGAGAGSGAASGAGAGAGAGAGTGSSGFGPYVAHGGYSGYEYAWSSESDFGTGSGAGAGSGAGAGSGAGAGSGAGAGSGAGAGSGAGAGSGAGAGYGAGVGAGYGAAYGAGAGAGYGAGAGSGAASGAGAGSGAGAGSGAGAGSGAGAGSGAGAGSGAGAGSGAGAGSGAGAGSGAGAGSGAGAGSGAGAGYGAGAGAGYGAGAGSGAGSGAGAGSGAGAGSGAGAGSGAGAGSGAGAGSGAGSGSGAGAGSGAGAGSGAGAGYGAGVGAGYGAGYGAGAGAGYGAGAGSGAGSGAGAGSGAGAGYGAGAGAGYGAGYGAGAGAGYGAGAGTGAGSGAGAGSGAGAGSGAGAGSGAGAGSGAGAGSGAGAGSGAGSGSGAGAGSGAGAGSGAGAGSGAGAGSGAGAGSGAGAGYGAGAGAGYGAGYGAGAGAGYGAGAGSGAGSGAGAGSGAGAGSGAGAGSGAGAGYGAGYGAGAGSGAASGAGAGAGAGAGTGSSGFGPYVAHGGYSGYEYAWSSESDFGTGSGAGAGSGAGAGAGAGAGSGAGAGYGAGVGAGYGAGYGAGAGAGYGAGAGSGTGSGAGAGSGAGAGYGAGVGAGYGAGAGSGAAFGAGAGAGAGSGAGAGSGAGAGSGAGAGSGAGAGSGAGAGYGAGYGAGVGAGYGAGAGSGAASGAGAGSGAGAGSGAGAGSGAGAGSGAGAGSGAGAGYGAGVGAGYGAGYGAGAGAGYGAGAGSGAASGAGAGSGAGAGAGSGAGAGSGAGAGSGAGAGSGAGSGAGAGSGAGAGSGAGAGYGAGAGSGAASGAGAGAGAGAGTGSSGFGPYVANGGYSGYEYAWSSESDFGTGSGAGAGSGAGAGSGAGAGSGAGAGSGAGAGYGAGVGAGYGAGYGAGAGAGYGAGAGSGAGSGAGAGSGAGAGSGAGAGSGAGAGSGAGAGSGAGAGSGAGAGYGAGAGSGAASGAGAGSGAGAGSGAGAGSGAGAGSGAGAGSGAGAGSGAGAGYGAGVGAGYGVGYGAGAGAGYGAGAGSGAGSGAGAGSGAGAGSGAGAGSGAGAGSGAGSGAGAGSGAGAGSGAGAGSGAGSGAGAGSGAGAGYGVGYGAGAGAGYGAGAGSGAGSGAGAGSGAGAGSGAGAGSGAGSGAGAGSGAGAGSGAGAGSGAGAGYGAGVGAGYGVGYGAGAGAGYGAGAGSGAGSGAGAGSGAGAGSGAGAGSGAGAGSGAGAGSGAGAGSGAGAGSGAGSGAGAGSGAGAGSGAGAGSGAGAGSGAGSGAGAGSGAGAGSGAGAGSGAGAGYGAGVGAGYGVGYGAGVGAGYGAGAGSGAASGAGAGSGAGAGAGSGAGAGSGAGAGSGAGAGSGAGAGSGAGAGSGAGAGYGAGYGAGVGAGYGAGAGVGYGAGAGAGYGAGAGSGAASGAGAGAGSGAGAGTGAGAGSGAGAGYGAGAGSGAASGAGAGAGAGAGTGSSGFGPYVANGGYSGYEYAWSSESDFGTGSGAGAGSGAGAGSGAGAGSGAGAGSGAGAGYGAGVGAGYGAGAGSGAGSGAGAGSGAGAGSGAGAGSGAGAGSGAGAGYGAGAGSGTGSGAGAGSGAGAGSGAGAGSGAGAGSGAGAGSGAGAGSGVGAGYGVGYGAGAGAGYGVGYGAGAGAGYGAGAGSGTGSGAGAGSGAGAGSGAGAGSGAGAGSGAGAGSGAGAGSGAGAGYGAGVGAGYGVGYGAGAGAGYGAGAGSGAGSGAGAGSGAGAGSGAGAGSGAGAGSGAGSGAGAGSGAGAGSGAGAGSGAGSGAGAGSGAGAGYGVGYGAGAGAGYGAGAGSGAGSGAGAGSGAGAGSGAGAGSGAGSGAGAGSGAGAGSGAGAGSGAGAGYGAGVGAGYGVGYGAGAGAGYGAGAGSGAGSGAGAGSGAGAGSGAGAGSGAGAGSGAGAGSGAGAGSGAGAGSGAGSGAGAGSGAGAGSGAGAGSGAGAGYGAGVGAGYGVGYGAGAGAGYGAGAGSGAASGAGAGAGAGAGTGSSGFGPYVANGGYSGYEYAWSSESDFGTGSGAGAGSGAGAGSGAGAGYGAGYGAGVGAGYGAGAGVGYGAGAGAGYGAGAGSGAASGAGAGAGAGAGSGAGAGSGAGAGAGSGAGAGYGAGYGIGVGAGYGAGAGVGYGAGAGAGYGAGAGSGAASGAGAGSGAGAGSGAGAGSGAGAGSGAGAGSGAGAGSGAGAGYGAGYGAGVGAGYGAGAGVGYGAGAGAGYGAGAGSGAASGAGAGAGAGAGAGSGAGAGSGAGAGSGAGAGSGAGAGSGAGAGSGAGAGSGAGAGSGAGAGSGAGAGYGAGVGAGYGAGYGGAGAGYGAGAGSGAASGAGAGSGAGAGSGAGAGSGAGAGSGAGAGSGAGAGYGAGAGSGAASGAGAGAGAGAGTGSSGFGPYVNGGYSGYEYAWSSESDFGTGSGAGAGSGAGAGSGAGAGYGAGVGAGYGAGYGAGAGAGYGAGAGSGAASGAGAGSGAGAGSGAGAGSGAGAGSGAGSGAGAGSGAGAGSGAGAGSGAGAGSGAGAGSGAGAGYGAGVGAGYGAGYGAGAGAGYGAGAGSGAASGAGAGSGAGAGAGSGAGAGSGAGAGSGAGAGSGAGAGSGAGAGSGAGSGAGAGSGAGAGYGAGYGAGVGAGYGAGAGVGYGAGAGAGYGAGAGSGAASGAGAGSGSGAGSGAGAGSGAGAGSGAGAGAGSGAGAGSGAGAGSGAGAGYGAGYGAGAGSGAASGAGAGAGAGAGTGSSGFGPYVANGGYSGYEYAWSSESDFGTGSGAGAGSGAGAGSGAGAGYGAGVGAGYGAGYGAGAGAGYGAGAGSGAGSGAGAGSGAGAGSGAGAGSGAGAGSGAGAGSGAGAGSGAGAGSGAGAGYGAGYGAGAGAGYGAGAGVGYGAGAGAGYGAGAGSGAGSGAGAGSGSGAGAGSGSGAGSGAGAGSGAGAGSGAGAGSGAGAGSGAGAGSGAGAGSGAGAGYGAGYGIGVGAGYGAGAGVGYGAGAGAGYGAGAGSGAASGAGAGSGAGAGSGAGAGSGAGAGSGAGAGSGAGAGSGAGAGSGAGAGSGAGAGSGAGAGYGAGAGVGYGAGAGSGAASGAGAGSGAGAGSGAGAGSGAGAGSGAGAGSGAGAGSGAGAGSGAGSGAGAGSGAGAGYGAGYGAGVGAGYGAGAGYGAGYGVGAGAGYGAGAGSGAGSGAGAGSGAGAGSGAGAGSGAGAGSGAGAGSGAGSGAGAGYGAGAGAGYGAGAGAGYGAGAGSGAASGAGAGAGAGSGAGAGSGAGAGSGAGSGAGAGSGAGAGYGAGAGSGAASGAGAGSGAGAGAGAGAGAGSGAGAGSGAGAGYGAGAGSGAASGAGAGAGAGTGSSGFGPYVANGGYSRREGYEYAWSSKSDFETGSGAASGAGAGAGSGAGAGSGAGAGSGAGAGSGAGAGGSVSYGAGRGYGQGAGSAASSVSSASSRSYDYSRRNVRKNCGIPRRQLVVKFRALPCVNCMRVKTFVILCCALQYVAYTNANINDFDEDYFGSDVTVQSSNTTDEIIRDASGAVIEEQITTKKMQRKNKNHGILGKNEKMIKTFVITTDSDGNESIVEEDVLMKTLSDGTVAQSYVAADAGAYSQSGPYVSNSGYSTHQGYTSDFSTSAAVGAGAGAGAAAGSGAGAGAGYGAASGAGAGAGAGAGAGYGTGAGAGAGAGYGAGAGAGAGAGYGAGAGAGAGAGYGAGAGAGAGAGYGAGAGAGAGAGYGAGAGAGAGAGYGAASGAGAGAGYGQGVGSGAASGAGAGAGAGSAAGSGAGAGAGTGAGAGYGAGAGAGAGAGYGAASGTGAGYGAGAGAGYGGASGAGAGAGAGAGAGAGAGYGTGAGYGAGAGAGAGAGAGAGYGAGAGAGYGAGYGVGAGAGYGAGYGAGAGSGAASGAGSGAGAGSGAGAGSGAGAGSGAGAGSGAGAGSGAGAGSGAGAGSGAGAGSGTGAGSGAGAGYGAGAGAGYGAGAGSGAASGAGAGSGAGAGSGAGAGSGAGAGSGAGAGSGAGAGYGAGAGAGYGAGAGAGYGAGAGVGYGAGAGSGAASGAGAGSGAGAGSGAGAGSGAGAGSGAGAGSGAGAGSGAGAGSGAGAGSGAGAGSGAGVGYGAGVGAGYGAGYGAGAGAGYGAGAGSGAASGAGAGAGAGAGTGSSGFGPYVANGGYSRSDGYEYAWSSDFGTGSGAGAGSGAGAGSGAGAGSGAGAGSGAGAGSGAGAGYGAGVGVGYGAGYGAGAGAGYGAGAGSGAASGAGAGSGAGAGSGAGAGSGAGAGSGAGAGSGAGAGSGAGAGSGAGAGSGAGAGSGAGAGSGAGVGSGAGAGSGAGAGVGYGAGAGVGYGAGAGSGAASGAGAGSGAGAGSGAGAGSGAGAGSGAGAGSGAGAGSGAGAGSGAGAGSGAGAGSGAGVGYGAGVGAGYGAGYGAGAGAGYGAGAGSGAASGAGAGSGAGAGSGAGAGSGAGAGSGAGAGSGAGAGSGAGAGSGAGAGSGAGAGSGAGAGSGAGAGSGAGAGYGAGAGAGYGAGYGAGAGAGYGAGAGSGAASGAGSGAGAGSGAGAGAGSGAGAGSGAGAGSGAGAGSGAGAGSGAGAGSGAGAGYGAGVGAGYGAGYGAGAGAGYGAGAGSGAASGAGAGSGAGAGSGAGAGSGAGAGSGAGAGSGAGAGSGAGAGSGAGVGYGAGYGAGAGAGYGAGAGSGAASGAGAGAGAGAGTGSSGFGPYVAHGGYSGYEYAWSSESDFGTGSGAGAGSGAGAGSGAGAGSGAGAGSGAGYGAGVGAGYGAGYGAGAGAGYGAGAGSGAGSGAGAGSGAGAGSGAGAGSGAGAGSGAGAGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGAGYGAGVGAGYGAGYGAGAGAGYGAGAGSGAGSGAGAGSGAGAGSGAGAGSGAGVGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGVGYGAGVGAGYGAGYGAGAGAGYGAGAGSGAASGAGAGAGAGAGTGSSGFGPYVANGGYSGYEYAWSSESDFGTGSGAGAGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGSGSGAGAGSGAGAGSGAGAGYGAGVGAGYGVGYGAGAGAGYGAGAGSGAASGAGAGAGAGAGTGSSGFGPYVAHGGYSGYEYAWSSESDFGTGSGAGAGSGAGAGSGAGAGSGAGAGSGAGAGSGAGAGSGAGAGYGAGVGAGYGAAYGAGAGAGYGAGAGSGAASGAGAGSGAGAGSGAGAGSGAGAGSGAGAGSGAGAGSGAGAGSGAGAGSGAGAGSGAGAGSGAGAGYGAGAGAGYGAGAGSGAGSGAGAGSGAGAGSGAGAGSGAGAGSGAGAGSGAGSGSGAGAGSGAGAGSGAGAGYGAGVGAGYGAGYGAGAGAGYGAGAGSGAGSGAGAGSGAGAGYGAGAGAGYGAGYGAGAGAGYGAGAGTGAGSGAGAGSGAGAGSGAGAGSGAGAGSGAGAGSGAGAGSGAGSGSGAGAGSGAGAGSGAGAGSGAGAGSGAGAGSGAGAGYGAGAGAGYGAGYGAGAGAGYGAGAGSGAGSGAGAGSGAGAGSGAGAGSGAGAGYGAGYGAGAGSGAASGAGAGAGAGAGTGSSGFGPYVAHGGYSGYEYAWSSESDFGTGSGAGAGSGAGAGAGAGAGSGAGAGYGAGVGAGYGAGYGAGAGAGYGAGAGSGTGSGAGAGSGAGAGYGAGVGAGYGAGAGSGAAFGAGAGAGAGSGAGAGSGAGAGSGAGAGSGAGAGSGAGAGYGAGYGAGVGAGYGAGAGSGAASGAGAGSGAGAGSGAGAGSGAGAGSGAGAGSGAGAGYGAGVGAGYGAGYGAGAGAGYGAGAGSGAASGAGAGSGAGAGAGSGAGAGSGAGAGSGAGAGSGAGSGAGAGSGAGAGSGAGAGYGAGAGSGAASGAGAGAGAGAGTGSSGFGPYVANGGYSGYEYAWSSESDFGTGSGAGAGSGAGAGSGAGAGSGAGAGSGAGAGYGAGVGAGYGAGYGAGAGAGYGAGAGSGAGSGAGAGSGAGAGSGAGAGSGAGAGSGAGAGSGAGAGSGAGAGYGAGAGSGAASGAGAGSGAGAGSGAGAGSGAGAGSGAGAGSGAGAGSGAGAGYGAGVGAGYGVGYGAGAGAGYGAGAGSGAGSGAGAGSGAGAGSGAGAGSGAGAGSGAGSGAGAGSGAGAGSGAGAGSGAGSGAGAGSGAGAGYGVGYGAGAGAGYGAGAGSGAGSGAGAGSGAGAGSGAGAGSGAGSGAGAGSGAGAGSGAGAGSGAGAGYGAGVGAGYGVGYGAGAGAGYGAGAGSGAGSGAGAGSGAGAGSGAGAGSGAGAGSGAGAGSGAGAGSGAGAGSGAGSGAGAGSGAGAGSGAGAGSGAGAGSGAGSGAGAGSGAGAGSGAGAGSGAGAGYGAGVGAGYGVGYGAGVGAGYGAGAGSGAASGAGAGSGAGAGAGSGAGAGSGAGAGSGAGAGSGAGAGSGAGAGSGAGAGYGAGYGAGVGAGYGAGAGVGYGAGAGAGYGAGAGSGAASGAGAGAGSGAGAGTGAGAGSGAGAGYGAGAGSGAASGAGAGAGAGAGTGSSGFGPYVANGGYSGYEYAWSSESDFGTGSGAGAGSGAGAGSGAGAGSGAGAGSGAGAGYGAGVGAGYGAGAGSGAGSGAGAGSGAGAGSGAGAGSGAGAGSGAGAGYGAGAGSGTGSGAGAGSGAGAGSGAGAGSGAGAGSGAGAGSGAGAGSGVGAGYGVGYGAGAGAGYGVGYGAGAGAGYGAGAGSGTGSGAGAGSGAGAGSGAGAGSGAGAGSGAGAGSGAGAGSGAGAGYGAGVGAGYGVGYGAGAGAGYGAGAGSGAGSGAGAGSGAGAGSGAGAGSGAGAGSGAGSGAGAGSGAGAGSGAGAGSGAGSGAGAGSGAGAGYGVGYGAGAGAGYGAGAGSGAGSGAGAGSGAGAGSGAGAGSGAGSGAGAGSGAGAGSGAGAGSGAGAGYGAGVGAGYGVGYGAGAGAGYGAGAGSGAGSGAGAGSGAGAGSGAGAGSGAGAGSGAGAGSGAGAGSGAGAGSGAGSGAGAGSGAGAGSGAGAGSGAGAGYGAGVGAGYGVGYGAGAGAGYGAGAGSGAASGAGAGAGAGAGTGSSGFGPYVANGGYSGYEYAWSSESDFGTGSGAGAGSGAGAGSGAGAGYGAGYGAGVGAGYGAGAGVGYGAGAGAGYGAGAGSGAASGAGAGAGAGAGSGAGAGSGAGAGAGSGAGAGYGAGYGIGVGAGYGAGAGVGYGAGAGAGYGAGAGSGAASGAGAGSGAGAGSGAGAGSGAGAGSGAGAGSGAGAGSGAGAGYGAGYGAGVGAGYGAGAGVGYGAGAGAGYGAGAGSGAASGAGAGAGAGAGAGSGAGAGSGAGAGSGAGAGSGAGAGSGAGAGSGAGAGSGAGAGSGAGAGSGAGAGYGAGVGAGYGAGYGGAGAGYGAGAGSGAASGAGAGSGAGAGSGAGAGSGAGAGSGAGAGSGAGAGYGAGAGSGAASGAGAGAGAGAGTGSSGFGPYVNGGYSGYEYAWSSESDFGTGSGAGAGSGAGAGSGAGAGYGAGVGAGYGAGYGAGAGAGYGAGAGSGAASGAGAGSGAGAGSGAGAGSGAGAGSGAGSGAGAGSGAGAGSGAGAGSGAGAGSGAGAGSGAGAGYGAGVGAGYGAGYGAGAGAGYGAGAGSGAASGAGAGSGAGAGAGSGAGAGSGAGAGSGAGAGSGAGAGSGAGAGSGAGSGAGAGSGAGAGYGAGYGAGVGAGYGAGAGVGYGAGAGAGYGAGAGSGAASGAGAGSGSGAGSGAGAGSGAGAGSGAGAGAGSGAGAGSGAGAGSGAGAGYGAGYGAGAGSGAASGAGAGAGAGAGTGSSGFGPYVANGGYSGYEYAWSSESDFGTGSGAGAGSGAGAGSGAGAGYGAGVGAGYGAGYGAGAGAGYGAGAGSGAGSGAGAGSGAGAGSGAGAGSGAGAGSGAGAGSGAGAGSGAGAGSGAGAGYGAGYGAGAGAGYGAGAGVGYGAGAGAGYGAGAGSGAGSGAGAGSGSGAGAGSGSGAGSGAGAGSGAGAGSGAGAGSGAGAGSGAGAGSGAGAGSGAGAGYGAGYGIGVGAGYGAGAGVGYGAGAGAGYGAGAGSGAASGAGAGSGAGAGSGAGAGSGAGAGSGAGAGSGAGAGSGAGAGSGAGAGSGAGAGSGAGAGYGAGAGVGYGAGAGSGAASGAGAGSGAGAGSGAGAGSGAGAGSGAGAGSGAGAGSGAGAGSGAGSGAGAGSGAGAGYGAGYGAGVGAGYGAGAGYGAGYGVGAGAGYGAGAGSGAGSGAGAGSGAGAGSGAGAGSGAGAGSGAGAGSGAGSGAGAGYGAGAGAGYGAGAGAGYGAGAGSGAASGAGAGAGAGSGAGAGSGAGAGSGAGSGAGAGSGAGAGYGAGAGSGAASGAGAGSGAGAGAGAGAGAGSGAGAGSGAGAGYGAGAGSGAASGAGAGAGAGTGSSGFGPYVANGGYSRREGYEYAWSSKSDFETGSGAASGAGAGAGSGAGAGSGAGAGSGAGAGSGAGAGGSVSYGAGRGYGQGAGSAASSVSSASSRSYDYSRRNVRKNCGIPRRQLVVKFRALPCVNC

SEQ ID NO: 2 - protein, 262 aa, Bombyx mori
MKPIFLVLLVATSAYAAPSVTINQYSDNEIPRDIDDGKASSVISRRWDYVDDTDKSIAILNVQEILKDMASQGDYASQASAVAQTAGIIAHLSAGIPGDACAAANVINSYTDGVRSGNFAGFRQSLGPFFGHVGQNLNLINQLVINPGQLRYSVGPALGCAGGGRIYDFEAAWDAILASSDSSFLNEEYCIVKRLYNSRNSQSNNIAAYITAHLLPPVAQVFHQSAGSITDLLRGVGNGNDATGLVANAQRYIAQAASQVHV

SEQ ID NO: 3 - protein, 620 aa, Drosophila melanogaster
MFKLLGLTLLMAMVVLGRPEPPVNSYLPPSDSYGAPGQSGPGGRPSDSYGAPGGGNGGRPSDSYGAPGQGQGQGQGQGGYAGKPSDTYGAPGGGNGNGGRPSSSYGAPGGGNGGRPSDTYGAPGGGNGGRPSDTYGAPGGGGNGNGGRPSSSYGAPGQGQGNGNGGRSSSSYGAPGGGNGGRPSDTYGAPGGGNGGRPSDTYGAPGGGNNGGRPSSSYGAPGGGNGGRPSDTYGAPGGGNGNGSGGRPSSSYGAPGQGQGGFGGRPSDSYGAPGQNQKPSDSYGAPGSGNGNGGRPSSSYGAPGSGPGGRPSDSYGPPASGSGAGGAGGSGPGGADYDNDEPAKYEFNYQVEDAPSGLSFGHSEMRDGDFTTGQYNVLLPDGRKQIVEYEADQQGYRPQIRYEGDANDGSGPSGPGGPGGQNLGADGYSSGRPGNGNGNGNGGYSGGRPGGQDLGPSGYSGGRPGGQDLGAGGYSNGKPGGQDLGPGGYSGGRPGGQDLGRDGYSGGRPGGQDLGASGYSNGRPGGNGNGGSDGGRVIIGGRVIGGQDGGDQGYSGGRPGGQDLGRDGYSSGRPGGRPGGNGQDSQDGQGYSSGRPGQGGRNGFGPGGQNGDNDGSGYRY

SEQ ID NO: 4 - protein, 575 aa, Drosophila melanogaster
MFKLLGLTLLMAMVVLGRPEPPVNSYLPPSDSYGAPGQSGPGGRPSDSYGAPGGGNGGRPSDSYGAPGQGQGQGQGQGGYAGKPSDTYGAPGGGNGNGGRPSSSYGAPGGGNGGRPSDTYGAPGGGNGGRPSDTYGAPGGGGNGNGGRPSSSYGAPGQGQGNGNGGRSSSSYGAPGGGNGGRPSDTYGAPGGGNGGRPSDTYGAPGGGNNGGRPSSSYGAPGGGNGGRPSDTYGAPGGGNGNGSGGRPSSSYGAPGQGQGGFGGRPSDSYGAPGQNQKPSDSYGAPGSGNGNGGRPSSSYGAPGSGPGGRPSDSYGPPASGSGAGGAGGSGPGGADYDNDIVEYEADQQGYRPQIRYEGDANDGSGPSGPGGPGGQNLGADGYSSGRPGNGNGNGNGGYSGGRPGGQDLGPSGYSGGRPGGQDLGAGGYSNGKPGGQDLGPGGYSGGRPGGQDLGRDGYSGGRPGGQDLGASGYSNGRPGGNGNGGSDGGRVIIGGRVIGGQDGGDQGYSGGRPGGQDLGRDGYSSGRPGGRPGGNGQDSQDGQGYSSGRPGQGGRNGFGPGGQNGDNDGSGYRY

SEQ ID NO: 5 - protein, 623 aa, Drosophila sechellia
MFKLLGLTLLMAMVVLGRPEPPVNSYLPPSDSYGAPGQSGAGGRPSDTYGAPGGGNGGRPSDSYGAPGQGQGQGQGQGGYGGKPSDSYGAPGGGNGNGGRPSSSYGAPGGGNGGRPSDTYGAPGGGNGGRPSDTYGAPGGGGNGNGGRPSSSYGAPGQGQGNGNGGRPSSSYGAPGGGNGGRPSDTYGAPGGGNGGRPSDTYGAPGGGNNGGRPSSSYGAPGGGNGGRPSDTYGAPGGGNGNGSGGRPSSSYGAPAQGQGGFGGRPSDSYGAPGQNQKPSDSYGAPGSGNGSAGRPSSSYGAPGSGPGGRPSDSYGPPASGSGAGGAGGSGPGGADYDNDEPAKYEFNYQVEDAPSGLSFGHSEMRDGDFTTGQYNVLLPDGRKQIVEYEADQQGYRPQIRYEGDANDGSGPSGPSGPGGPGGQNLGADGYSSGRPGNGNGNGNGGYSSGRPGGQDLGPSGYSGGRPGGQDLGAGGYSNVKPGGQDLGPGGYSGGRPGGQDLGRDGYSGGRPGGQDLGAGAYSNGRPGGNGNGGSDGGRVIIGGRVIGGQDGGDQGYSGGRPGGQDLGRDGYSSGRPGGRPGGNGQDSQDGQGYSSGRPGQGGRNGFGPGGQNGDNDGSGYRY

SEQ ID NO: 6 - protein, 619 aa, Drosophila erecta
MFKLLGLTLLMAMVVLGRPEPPVNSYLPPSDSYGAPGQSGPGGRPSDSYGAPGGGNGGRPSDSYGAPGLGQGQGQGQGQGGFGGKPSDSYGAPGAGNGNGGRPSSSYGAPGAGNGGRPSDTYGAPGGGSGGRPSDTYGAPGGGNGNGNGGRPSSSYGAPGQGQGNGNSGRPSSSYGAPGAGNGGRPSDTYGAPGGGNGGRPSSSYGAPGAGNGGNGGRPSDTYGAPGGGNGNGNGNGNGSGGRPSSSYGAPGQGQGGFGGRPSDSYGAPGQNQKPSDSYGAPGSGSGSGNGNGGRPSSSYGAPGSGPGGRPSDSYGPPASGSGAGGAGGSGPGGADYDNDEPAKYEFNYQVEDAPSGLSFGHSEMRDGDFTTGQYNVLLPDGRKQIVEYEADQQGYRPQIRYEGDANDGSGPSGPGGQNLGADGYSSGRPGNGNGNGNGGYSGGRPGGQDLGPSGYSGGRPGGQDLGAGGYSNGRPGGQDLGPSGYSGGRPGGQDLGPSGYSGGRPGGQDLGAGGYSNGRPGGNGNGNGGADGGRVIIGGRVIGGQDGGDQGYSGGRPGGQDLGRDGYSSGRPGGRPGANGQDNQDGQGYSSGRSGKGGRNSFGPGGQNGDNDGSGYRY

SEQ ID NO: 7 - protein, 609 aa, Drosophila simulans
MFKLLGLTLLMAMVVLGRPEPPVNSYLPPSDSYGAPGQSGVPAGRPSDSYGAPGQGQGQGQGQGGYGGKPSDSYGAPGGGNGNGGRPSSSYGAPGGGNGGRPSDTYGAPGGGNGGRPSDTYGAPGGGGNGNGGRPSSSYGAPGQGQGNGNGGRPSSSYGAPGGGNGGRPSDTYGAPGGGNGGRPSDTYGAPGGGNNGGRPSSSYGAPGGGNGGRPSDTYGAPGGGNGNGSGGRPSSSYGAPGQGQGGFGGRPSDSYGAPGQNQKPSDSYGAPGSGNGSGGRPSSSYGAPGSGPGGRPSDSYGPPASGSGAGGAGGSGPGGADYDNDEPAKYEFNYQVEDAPSGLSFGHSEMRDGDFTTGQYNVLLPDGRKQIVEYEADQQGYRPQIRYEGDANDGSGPSGPSGPGGPGGQNLGADGYSSGRPGNGNGNGNGGYSGGRPGGQDLGPSGYSGGRPGGQDLGAGGYSNGKPGGQDLGPGGYSGGRPGGQDLGRDGYSGGRPGGQDLGAGGYSNGRPGGNGNGGSDGGRVIIGGRVIGGQDGGDQGYSGGRPGGQDLGRDGYSSGRPGGRPGGNGQDSQDGQGYSSGRPGQGGRNGFGPGGQNGDNDGSGYRY

SEQ ID NO: 8 - protein, 622 aa, Drosophila yakuba
MFKLLGLTLLMAMVVLGRPEPPVNSYLPPSDSYGAPGQSGPGGRPSDAYGAPGGGNGGRPSDSYGAPGVGQGQGQGQGQGGFGGKPSDSYGAPGGGNGNGGRPSSSYGAPGGGNGGRPSDTYGAPGGGSGGRPSDSYGAPGGGNGNSGRPSSSYGAPGQGQGNGNGGRPSGSYGAPGGGNGGRPSDTYGAPGGGNGGRPSSSYGAPGGGNGNGGRPSDSYGAPGGGNGNGNGSGGRPSSSYGAPGQGQGGFGGRPSDSYGAPGQNQKPSDTYGAPGNGNGNGNGGRPSSSYGAPGTGPGGRPSDSYGPPASGSGAGGAGGGSGPGGADYDNDEPAKYEFNYQVEDAPSGLSFGHSEMRDGDFTTGQYNVLLPDGRKQIVEYEADQQGYRPQIRYEGDANDGSGPSGPSGPGGAGGPGGQNLGADGYSSGRPGNGNGNGNGGYPGGRPGGQDLGPSGYSGGRPGGQDLGPGGYSNGRPGGQDLGPSGYSGGRPGGQDLGPNGYSGGRPGGQDLGPGGYSNGRPGGNGNGNGNGGSDGGRVIIGGRVIGGQDGGDQGYSGGRPGGQDLGRDGYSGGRPGGRPGANGQDNQDGQGYSSGRPGQGGRNGFGPGGQNGDNDGSGYRY

SEQ ID NO: 9 - protein, 621 aa, Drosophila pseudoobscura
MFKLLGLTLLCTAVLARPEPPVNSYLPPSDSYGAPGQSGPGAQGGGPGGRPTDSYGPPGLGQGQGQGQGQAQGGFGGKPSDSYGAPGVGGNGNGGGRPSSSYGAPGNANGGGRPSSSYGAPGQGQGQGGRPSDSYGAPGASGNGNGNGGGRPSSSYGAPGQGQGGRPSDSYGAPGASGNGNGNGGGRPSSSYGAPGQGQGGRPSDSYGAPGASGNGNGNGGSRPSSNYGAPGQGQGGRPSDSYGAPGASGNGNGNGGGRPSSSYGAPGAGSGGRPSDSYGPPASGAGAGGGSGGPGGSGADYDNDEPAKYEFNYQVEDAPSGLSFGHSEMRDGDFTTGQYNVLLPDGRKQIVEYEADQQGYRPQIRYEGEANDGGASGPGGQNLGADGYSSGRPGGNGNGQNGYSGGRPGGQDLGRDGYSGGRPGGQDLGQNGYSGGRPGGQDLGASGYSGGRPGGQDLGQNGYSGGRPGGQDLGAGGYSGGRPGGQDLGQNGYSSGRPGGNGGNAGSDGGRVIIAGRVTGPDGSDQGQGFSGGRPGGNSNGGRQISGQDLGRDGYSGGRPGGQDLGAGGYSGGRPGGQDLGQNGYSSGKPQGQGQGQGQGRNGFGPGGQNGDNDGSGYRY

SEQ ID NO: 10 - protein, 621 aa, Drosophila persimilis
MFKLLGLTLLCTAVLARPEPPVNSYLPPSDSYGAPGQSGPGAQGGGPGGRPTDSYGPPGLGQGQGQGQGQGQAQGGFGGKPSDSYGAPGVGGNGNGGGRPSSSYGAPGNANGGGRPSSSYGAPGQGQGQGGRPSDSYGAPGASGNGNGNGGGRPSSSYGAPGQGQGGRPSDSYGAPGASGNGNGNGGGRPSSSYGAPGQGQGGRPSDSYGAPGASGNGNGNGGSRPSSNYGAPGQGQGGRPSDSYGAPGASGNGNGNGGGRPSSSYGAPGAGSGGRPSDSYGPPASGAGAGGGSGGPGGSGADYDNDEPAKYEFNYQVEDAPSGLSFGHSEMRDGDFTTGQYNVLLPDGRKQIVEYEADQQGYRPQIRYEGEANDGGASGPGGQNLGADGYSSGRPGGNGNGQNGYSGGRPGGQDLGRDGYSGGRPGGQDLGQNGYSGGRPGGQDLGASGYSGGRPGGQDLGQNGYSGGRPGGQDLGAGGYSGGRPGGQDLGQNGYSSGRPGGNGGNAGSDGGRVIIAGRVTGPDGSDQGQGFSGGRPGGNSNGGRQIGGQDLGRDGYSGGRPGGQDLGAGGYSGGRPGGQDLGQNGYSSGKPQGQGQGQGRNGFGPGGQNGDNDGSGYRY

SEQ ID NO: 11 - protein, 589 aa, Drosophila mojavensis
MFKLFGIMLLTATVLARPEPPVNSYLPPSPGDSYGAPGQGQGQGQGGFGGRPSDSYGAPGAGNGNGGGRPSSSYGAPGQGQGRPSDSYGAPGAGNGNGGGRPSSSYGAPGQGQGGLGGRPSSSYGAPGSGNGNGNGRPSSSYGAPGQGQGGKPSDSYGAPGLGNGNGNGNGRPSSSYGAPGQGQGSFGGKPSDTYGAPGAGNANGNGRPSSSYGAPGQGQGGFGGKPSDSYGAPGAGNGNGGGRPSSSYGAPGQGQGQGGFGGRPSDSYGPPASGAGSAGAGGPGGAGGDYDNDEPAKYEFNYQVEDPPSGLSFGHSEMRDGDFTTGQYNVLLPDGRKQIVEYEADQQGYRPQIRYEGDANGGAGGAGGPGGQDLGQNGYSSGRPGGQDLGQGGYSGGRPGGQDLGQNGYSGGRPGGQDLGQNGYSGGRPGGQDLGQNGYSGGRPGGQDLGQNGYSSGRPGGQDLGQNGYSGGRPGGQDLGQNGYSGGRPGGQDLGQNGYSSGRPGGNGGSDGGRVIIGGRVIGQDAGDGQGYSSGRPNGQDGGFGQDNVDGRGYSSGKPGQGRNGNGNGSSFGPGGQNGDNDGSGYRY

SEQ ID NO: 12 - protein, 633 aa, Drosophila virilis
MFKLFGLTLLLTAAVLARPEPPVNSYLPPSPGDSYGAPGQGQGQGQGGFGGKPSDSYGAPGAGNGNGNGRPSSSYGAPGQGQGQGGFGGKPSDSYGAPGAGNGNGNGRPSSSYGAPGQGQGQGGFGGRPSSSYGAPGQGQGGFGGKPSDTYGAPGAGNGNGRPSSSYGAPGQGQGGIGGKPSDSYGAPGAGNGNGNGRPSSSYGAPGQGQGGFGGKPSDTYGAPGAGNGNGRPSSSYGAPGQGQGGFGGKPSDTYGAPGAGNGNGNGRPSSSYGAPGQGQGGFGGKPSDTYGAPGAGNGNGRPSSSYGAPGQGQGQGGFGGKPSDSYGPPASGAGAGGAGGPGAGGGGDYDNDEPAKYEFNYQVEDAPSGLSFGHSEMRDGDFTTGQYNVLLPDGRKQIVEYEADQQGYRPQVRYEGDANGNGGPGGAGGPGGQDLGQNGYSSGRPGGQDLGQGGYSNGRPGGQDLGQNGYSGGRPGGQDLGQNGYSGGRPGGQDLGQNGYSGGRPGGQDLGQNGYSGGRPGGQDLGQNGYSGGRPGGQDLGQNGYSGGRPGGNGGSDGGRVIIGGRVIGQDGGDGQGYSSGRPNGQDGGFGQDNTDGRGYSSGKPGQGRNGNGNSFGPGGQNGDNDGSGYRY

SEQ ID NO: 13 - protein, 836 aa, Drosophila grimshawi
MSSSYAVLFGVVAAVFIVVVSPPDVAAAKREAPLNNAYLPPSSGSRPSSKYGAPPPSSYLPPASGPAPSFNSRPAPSSSYSAPGASSGGPYPASNHHKPSSSYGPPSRPAPAPSSSYGAPPSRPSQSYGPPPPRKHHSSRRPSSSYGPPKSSAPSSSYGAPAPPAPPSSKYGPPKSPSSSYGAPSRPSSSYGAPSVNSFVPLPSAPSTNYGAPSKTQVLGSSGYTSGPGAPSAPSSSYGAPSSGSSSFHPISPPSSKYGAPSGGSSSSSSSFSTHPSFSSPSSSYSAPSPSANSGGSYPAAPSSSYGAPSKGSSSGGPYPAAPSSSYGAPISKPSSSYSAPSPGASSGGPYPAAPSSSYGAPSSAPSSSYSAPSHGSNSGGPYPAAPSSSYGAPKRAPSSSYSAPSPGASSGGPYPAAPSSSYGAPSSAPSSSYSAPSHGSNSGGPYPAAPSSSYGAPKRAPSSSYSAPSSGASSGGPYPAAPSSSYGAPSTGPSSSYSAPSHEASSGGPYPAAPSSSYGAPSAPAPSSSYSAPSHGSSSGGPYPSAPSSSYGAPSSGPSSSYSAPSLGASSGGPYPAAPSSSYGAPSAPAPSSSYSAPSHGSNSGGPYPAAPSSSYGAPSSGPSSSYSAPSHGSSSGGPYPAAPSSSYGAPSSGPSSSYSAPSHGSSSGGPYPSAPSSSYGAPSTGPSSSYSAPAPAAPSSSYGAPPSSSYSAPSGGGSSGGPYPAAPPSSSYGAPSSSHSSGSFSSHGGSSFGSSFASSSASSSSSSGGGFSTGPLSSYSAPAAPSSSYGAPSEPSSSYGAPSQDSGYSYSAPSQVPETIQQGYSSNGGYTYRK

SEQ ID NO: 14 - protein, 605 aa, Drosophila grimshawi
MFKLFGLMLLLTTAVLARPEPPVNTYLPPTPGDSYGAPGQGQGQAQGGRPSSSYGAPGQGQGQGQPGFGGKPSDSYGAPAAGNGNGNGNGRPSSSYGAPGQSQGQNGFGGRPSSSYGAPGQGAAGNGNGNGNGRPSSSYGAPGQGQGQAGRPSDSYGAPAGGNGNGNGNGRPSSSYGAPGQGQGQGGFGGASGAGNGNGNVRPSSSYGAPGQGQGGRPSDSYGAPGAGNGNGRPSSSYGAPGQAQGGFGGKPSDSYGAPGAGAPGQGQGTGGFGGGKPSDSYGPPASGAGAGGGPGGAGGAGGDYNNDEPAKYEFNYQVEDAPSGLSFGHSEMRDGDFTTGQYNVLLPDGRKQIVEYEADQQGYRPQIRYEGDANGAGGAGGAGGAGGQDLGQAGYSSGRPGGQDLGQNGYSGGRPGGQDLGQNGYSSGRPGGQNGYSGGRPGGQDAAQGGYSGGRPGGQDLGQNGYSGGRPGGQDLGQNGYSGGRPGGQDLGQNGYSGGRPGGQDLGPNGYSSGRPGGNGNDNGNGGSDGGRVIIRGRVTGQDGGDGQGYSGGRPSGQNGGNGQDNIDGRGYSSGRPGQGQGQGRNGFGPGGQNGDNDGSGYRY

SEQ ID NO: 15 - protein, 605 aa, Drosophila willistoni
MFKLLGLMLLVTAVLARPEPPVNSYLPPSDSYGAPDANGGGGSGGRPSDSYGAPGNGNGNNGGGRPSSSYGAPGQGGQGQGGFGGRPSDSYGAPGNGNNGGGRPSSSYGAPGQGGPGGGLGGKPSDTYGAPGNGNNGGGRPSSSYGAPGQGGQGQGGIGGRPSDSYGAPGNGNNGGGRPSSSYGAPGQGGPGGKPSDTYGAPGNGNNGGGRPSSSYGAPGQGGQGQGGFGGRPSDSYGAPGNGNGGGRPSSSYGAPGQGGQGGGLGGKPSDTYGAPGNGNNGGGRPSSSYGAPGQGGQGQGGFGGRPSDSYGPPASGAGAGGGAGGNGNGADYDNDEPAKYEFNYQVEDAPSGLSFGHSEMRDGDFTTGQYNVLLPDGRKQIVEYEADQQGYRPQIRYEGDANDGSGSGSGTGPGGQSLGADGYSSGRPGNGNGGGGYSGGRPGGQDLGQNGYSSGRPGGNGNGNGDGGYPGGRPGGQDLGQGGYSSGRPGSNGNGNGGDGGRVIIGGRVIGQDNNDGQGYSGGRPGGQGQDFGAGGYSSGRPGGQGQDLNGQNGYSSGRPGGRSNDQDNDGQGYSSGKPGQGRNGNGFGPGGQNGDNDGSGYRY

SEQ ID NO: 16 - protein, 587 aa, Drosophila ananassae
MFKLLGLTLLMAVMVLGRPEPPVNSYLPPSDSYGAPGQSGPGGRPSDSYGAPGGGSGGRPSDSYGAPGLGGGQGGGFGGKPSDSYGAPGNGNGNGNGNGGRPSSSYGAPGLGGGQGGGFGGKPSDSYGAPGSGNGNGGRPSSSYGAPGQGQGGYSNGGNGNGNGNGGRPSSTYGAPGQGRPSDTYGAPGQGGNGGRPSDSYGAPGQGGNGGRPSDSYGAPGQGGNGGRPSSSYGAPGQGQGGQGGFGGRPSDTYGAPGQNGNGGRPSSSYGAPGSGPGGRPSDSYGPPASGAGAGGAGGSGPGLPGGGGADYDNDEPAKYEFNYQVEDAPSGLSFGHSEMRDGDFTTGQYNVLLPDGRKQIVEYEADQQGYRPQIRYEGDANDGSGASGPGGQNLGPDGYSNGRPGNGNGNGNGNGNGGYSGGRPGGQDLGPGGYSNGRPGGQDFGPSGYSGGRPGGQDLGAGGYSNGRPGGQDFGPGGYSSGRPGGNGNGNGNGNGNGNGDGRVIIGGRVIGGQDGGDQGYSGGRPGGQDLGRDGYSSGRPGGGRSNGQDNQDGQGYSSGKPGGQGRNGFGPGGQNGDNDGSGYRY

SEQ ID NO: 17 - protein, 805 aa, Culex quinquefasciatus
MRMHHKLTALAVLCCALGPAFAASVTKREAPLPGGSYLPPSNGGGAGGYPAAGPPSGSYGPPSNGNGNGNGAGGYPSAPSQQYGAPAGGAPSQQYGAPSNGNGGAGGYPSAPSQQYGAPNGNGNGGFGGRPQAPSQQYGAPSNGNGGARPSQQYGAPNGGNGNGRPQTPSSQYGAPSGGAPSSQYGAPSGGAPSQQYGAPNGGNGNGRPQTPSSQYGAPSGGAPSQQYGAPNGGNGNGRPQTPSSQYGAPSGGAPSSQYGAPSGGAPSSQYGAPAGGAPSSQYGAPAGGAPSSQYGAPAGGAPSSQYGAPAGGAPSSQYGAPAGGAPSSQYGAPSSQYGAPAGGAPSSQYGAPAGGAPSSQYGAPSGGAPSSQYGAPSGGAPSSQYGAPAGGAPSSQYGAPSGGAPSSHMALHLVVPHLSNTVPRKTDSRVKDHQPLATESQLDHLTETDLEDAHRASMVPQPPEETETEVAHRASTVPQLREETETVDTATVMVDALQASMVPQLQEATATVDALQASMVPQLQVVTATVDAHQANTVPQLQVETETDAQASRTEPQVPVLLLPNMALLLLLRLSMVPQLQVVTEMVDAHQANTVPQLQVETETDAQASRTELQVPVLLLPNMALLLLLRLSMVPQLQVVTETVDAHQANTVPQLQVETETDAQASRTAPQVPVLLLPNMALLLLPRLSTVLLRLHRRNMVPRATVTATAAAPPETATRRARSPPAASRPTVEAAAAPDTARADPTVDHPSRPPSLSHTLKAEATTTKLGLRTKPQTLLRTINGIPRTASHAPQMNMCLKTCMI

SEQ ID NO: 18 - protein, 567 aa, Tribolium castaneum
MDKHFLVLVLTWSAFVRAEPPVNSYLPPSQNGGPSSTYGPPGFQPGTPLGGGGNGGHPPSQGGNGGFGGRHPDSDQRPGTSYLPPGQNGGAGRPGVTYGPPGQGGGQNGGGPSSTYGPPGQGGNGFGGGQNGGRLSSTYGPPGQGGNGFGGGQNGGRPSSTYGPPGQGGNGFGGGQNGGRPSSTYGPPGQGGNGFGGGQNGGRPSSTYGPPGQGGNGFGGGQNGGRPSSTYGPPGQGGNGFGGGQNGGRPSSTYGPPGQGGNGFGGGQNGGKPSSTYGPPGQGGNGFGGGQNGGRPSSTYGPPGQGGNGNGGGHNGQRPGGSYLPPSQGGNGGYPSGGPGGYPSGGPGGNGGYGGEEESTEPAKYEFEYQVDDDEHNTHFGHQESRDGDKATGEYNVLLPDGRKQVVQYEADSEGYKPKISYEGGNGNGGYPSGGPGGAGNGGYPSGGPQGGNGGYPSGGPQGGNGGYPSGGPQGGNGGYPSGGPQGGNGGYPSGGPQGGNGGYPSGGPQGGNGGYPSGGPQGGNGGYPSGGPQGGNGGYTSGGPQGGNGGYPSGGPQGGNGGSGPY

SEQ ID NO: 19 - protein, 467 aa, Tribolium castaneum
MSSFKLVSCTLALCCLLNGSLGGQLTKRDAPLSGGYPSGGPANSYLPPGGASQPSGNYGAPSGGFGGKSGGFGGSGGFGGAPSQSYGAPSGGFGGSSSFGKSGGFGGAPSQSYGAPSGGFGGSSSFGKSSGGFGGAPSQSYGAPSGGFGGSSSFGKSGGFGGAPSQSYGAPSGGFGGSSSFGKSGGFGGAPSQSYGAPSGGFGGKSSSFSSAPSQSYGAPSGGFGGKSGGFGGAPSQSYGAPSGGFGGKSGGFGGAPSQSYGAPSGGFGGSSSFGKSGGFGGAPSQSYGAPSGGFGGSSSFGKSSGFGHGSGAPSQSYGAPSRSQPQSNYLPPSTSYGTPVSSAKSSGSFGGAPSQSYGAPSQSHAPSQSYGAPSRSFSQAPSQSYGAPSQGHAPAPQQSYSAPSQSYGAPSGGFGGGHGGFGGQGQGFGGGRSQPSQSYGAPAPSQSYGAPSAGGQQYASNGGYSY

SEQ ID NO: 20 - protein, 492 aa, Apis mellifera
MYPYLNQHPTLPPPVFSTSSPIAQTFCIQDMSVCTHWTFRSSHPFKFRSFRLQIGLAVFVLALTLVRSEPPVNSYLPPSGNGNGGGGGGSSNVYGPPGFDGQNGIGEGDNGRNGISNSYGVPTGGNGYNGDSSGNGRPGTNGGRNGNGNGRGNGYGGGQPSNSYGPPSNGHGGNGAGRPSSSYGAPGGGNGFAGGSNGKNGFGGGPSSSYGPPENGNGFNGGNGGPSGLYGPPGRNGGNGGNGGNGGRPSGSYGTPERNGGRLGGLYGAPGRNGNNGGNGYPSGGLNGGNGGYPSGGPGNGGANGGYPSGGSNGDNGGYPSGGPNGNGNGNGGYGQDENNEPAKYEFSYEVKDEQSGADYGHTESRDGDRAQGEFNVLLPDGRKQIVEYEADQDGFKPQIRYEGEANSQGYGSGGPGGNGGDNGYPSGGPGGNGYSSGRPNGGSDFSDGGYPSTRPGGENGGYRNGNNGGNGNGGYPSGNGGDAAANGGYQY

SEQ ID NO: 21 - protein, 344 aa, Apis mellifera
MRPGKSWAPLAATVLATALLLQPIHADAPISGSYLPPSTSYGTPNLGGGGPSSTYGAPSGGGGGRPSSSYGAPSSTYGAPSSTYGAPSNGGGRPSSTYGAPSNGGGRPSSSYGAPSSSYGAPSSTYGAPSNGGGRPSSSYGAPSFGGGGGFGGGNGLSTSYGAPSRGGGGGGGSISSSYGAPTGGGGGGPSTTYGAPNGGGNGYSRPSSTYGTPSTGGGSFGGSGGYSGGGGGYSGGGNGYSGGGGGGYSGGNGGGYSGGGNGGGYSGGNGGGYSGGGGGGYSGGGGGGYSGGGNGYSGGGGGGYSGGNGGYSGGNGGYSGGGGGYSGGGGGGQSYASNGGYQY

SEQ ID NO: 22 - protein, 752 aa, Nasonia vitripennis
MEKVICLIAVLTLCAARPEPPVNSYLPPGQGGGFGGGRPSGASPSDQYGPPDFQGAGGRGGQAAGGNFGGGGNGFGGAPSSSYGPPGFGSNEPNKFSGAGGGGAGRPQDSYGPPAGGNGFAGSAGAGNSGRPGGAAAGGRPSDSYGPPQGGGSGFGGGNAGRPSDSYGPPSAGGGGFGGGSPGGGFGGGSPGGGFGGGNQGAPQSSYGPPASGFGGQGGAGQGRPSDSYGPPGGGSGGRPSQGGNGFGGGNAGRPSDSYGPPAAGGGGFGGNAGGNGGGNGFGGGRPSGSPGGFGGQGGGGRPSDSYLPPSGGSGFGGGNGRQPGGFGQQGGNGAGQQNGGGGAGRPSSSYGPPSNGNGGGFSGQNGGRGSPSSGGGFGGAGGSPSSSYGPPAGGSGFGNNGGAGGRPSSSYGPPSSGQGGQGGRPSSSYGPPSNGNGGFGGGNGGRPSSNGYPQGQGNGNGGFGGQGGNGGRPSSSYGPPGGDSGYPSGGPSDSYGPPPSGAVNGNGNGYSSGGPGGNGLDEGNDEPAKYEFSYEVKDDQSGSNFGHTEMRDGDRAQGEFNVLLPDGRKQIVEYEADQDGFKPQIRYEGEANTGAGGAGGYPSGGGGDSGYPSGPSGAGGNAGYPSGGGGGAGGFGGNGGGSNGYPSGGPSGGQGQFGGQQGGNGGYPSGPQGGSGFGGGSQGSGSGGYPSGGPGGNGGNNNFGGGNAGYPSGGPSGGNGFNQGGQNQGGSGGGYPSGSGGDAAANGGYQY

SEQ ID NO: 23 - protein, 545 aa, Camponotus floridanus
MTRSEPPVNSYLPSRTGISGANDGQADLSTQYGTPDFGNGGNANRNGGATSFSGPGGNGAGNGPSKLYDAPIGGNARVNGLGQSRRNGFGNGQSSSYSASSFGDFSETGGNVRPSSSYGVPIANGNNGDGFRNGDNGDKPSINYGVPGINGNNGDRNRGNGERPSTNYGAPGANGNHGGGSSGNNNNGRPSTSYGVPANGNTNGKNHFNGGSNGNGGKLSSNYESPNVPKINGFGTNGGLSSSYGPPDRNGHGNNGYPSESPTRNGEGFRNGGANGYPSGGGTNGHVGNFENGGGSFKNEGRGNGGYNDNAQEESTEPAKYEFSYEVKDEQSGSNYGHKETRNGDHAQGEFNVLLPDGRKQIVEYEADQDGFKPQIRYEGEANTGGGYSSGGPNGNNDGYSSGRPDSKSGGFADNSGFNGGGTNGYPNGSPGEGKPNGFNGGGNGYQSGKSAGQSFSRDNDNNLNGNIGGYFSNAPSNHIGDNADIGNNRQNAGPVLGVTDLPERVAPGSRVCPTRTRKDKTDTRKFYCDLLAHLQGSKEIQVSR

SEQ ID NO: 24 - protein, 4 aa, Artificial Sequence
  Resilin-like polypeptide
YGAP

SEQ ID NO: 25 - protein, 11 aa, Artificial Sequence
  Resilin-like polypeptide
AQTPSSQYGAP

SEQ ID NO: 26 - protein, 15 aa, Artificial Sequence
  Resilin-like polypeptide
GGRPSDSYGAPGGGN

SEQ ID NO: 27 - protein, 13 aa, Artificial Sequence
  Resilin-like polypeptide
GYSGGRPGGQDLG

SEQ ID NO: 28 - protein, 5 aa, Artificial Sequence
  Resilin-like polypeptide
PGGGN

SEQ ID NO: 29 - protein, 9 aa, Artificial Sequence
  Resilin-like polypeptide
PGGGNGGRP

SEQ ID NO: 30 - protein, 15 aa, Artificial Sequence
  Resilin-like polypeptide
SDTYGAPGGGNGGRP

SEQ ID NO: 31 - protein, 24 aa, Artificial Sequence
  Resilin-like polypeptide
PGGGNGGRPSDTYGAPGGGNGGRP

SEQ ID NO: 32 - protein, 62 aa, Artificial Sequence
  Resilin-like polypeptide
SMSDTYGAPGGGNGGRPSDTYGAPGGGNGGRPSDTYGAPGGGNGGRPSDTYGAPGGGNGGRP

SEQ ID NO: 33 - protein, 50 aa, Artificial Sequence
  Resilin-like polypeptide
PGGGNPGGGNPGGGNPGGGNPGGGNPGGGNPGGGNPGGGNPGGGNPGGGN

SEQ ID NO: 34 - protein, 272 aa, Artificial Sequence
  Resilin-like polypeptide
GGGDQKGGRPSDSFGAPGGGNGGRPSDSFGAPGGGNGGRPSDSFGAPGGGNGGKGGRPSDSFGAPGGGNGGRPSDSFGAPGGGNGGRPSDSFGAPGGGNGGKGGGRGDSPAEDLGDQKGGRPSDSFGAPGGGNGGRPSDSFGAPGGGNGGRPSDSFGAPGGGNGGKGGRPSDSFGAPGGGNGGRPSDSFGAPGGGNGGRPSDSFGAPGGGNGGKGGGRGDSPAEDLGPQGIWGQGGRGGCKAAKRPKAAKDKQTKGEDLGDPMASMTGGQQM

SEQ ID NO: 35 - protein, 570 aa, Homo sapiens
MAGLTAAAPRPGVLLLLLSILHPSRPGGVPGAIPGGVPGGVFYPGAGLGALGGGALGPGGKPLKPVPGGLAGAGLGAGVGGAFAGIPGVGPFGGPQPGVPLGYPIKAPKLPGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGVAARPGFGLSPIFPGGACLGKACGRKRK

SEQ ID NO: 36 - protein, 705 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 37 - protein, 730 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGVGTPAAAAAKAAAKAAQFALLNLAGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 38 - protein, 692 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAAPSVPGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGVAARPGFGLSPIFPGGACLGKACGRKRK

SEQ ID NO: 39 - protein, 700 aa, Homo sapiens
MAGLTAAAPRPGVLLLLLSILHPSRPGGVPGAIPGGVPGGVFYPALGPGGKPLKPVPGGLAGAGLGAGLGAFPAVTFPGALVPGGVADAAAAYKAAKAGAGLGGVPGVGGLGVSAAPSVPGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 40 - protein, 381 aa, Homo sapiens [sequence omitted]

SEQ ID NO: 41 - protein, 711 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAAPSVPGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGVAARPGFGLSPIFPGGACLGKACGRKRK

SEQ ID NO: 42 - protein, 643 aa, Homo sapiens
MAGLTAAAPRPGVLLLLLSILHPSRPGGVPGAIPGGVPGGVFYPGAGLGALGGGALGPGGKPLKPGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGPFGGPQPGVPLGYPIKAPKLPGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGAEAQAAAAAKAAKYGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 43 - protein, 658 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGLVPGVGVAPGVGVAPGVGVAPGVGLAPGVGVAPGVGVAPGVGVAPGIGPGGVAGAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGVAARPGFGLSPIFPGGACLGKACGRKRK

SEQ ID NO: 44 - protein, 724 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 45 - protein, 677 aa, Homo sapiens
MAGLTAAAPRPGVLLLLLSILHPSRPGGVPGAIPGGVPGGVFYPALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGVAARPGFGLSPIFPGGACLGKACGRKRK

SEQ ID NO: 46 - protein, 617 aa, Homo sapiens
MAGLTAAAPRPGVLLLLLSILHPSRPGGVPGAIPGGVPGGVFYPALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGAEAQAAAAAKAAKYGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGVAARPGFGLSPIFPGGACLGKACGRKRK

SEQ ID NO: 47 - protein, 747 aa, Bos taurus
MRSLTAAARRPEVLLLLLCILQPSQPGGVPGAVPGGVPGGVFFPGAGLGGLGVGGLGPGVKPAKPGVGGLVGPGLGAEGSALPGAFPGGFFGAGGGAAGAAAAYKAAAKAGAAGLGVGGIGGVGGLGVSTGAVVPQLGAGVGAGVKPGKVPGVGLPGVYPGGVLPGAGARFPGIGVLPGVPTGAGVKPKAQVGAGAFAGIPGVGPFGGQQPGLPLGYPIKAPKLPAGYGLPYKTGKLPYGFGPGGVAGSAGKAGYPTGTGVGPQAAAAAAKAAAKLGAGGAGVLPGVGVGGPGIPGAPGAIPGIGGIAGVGAPDAAAAAAAAAKAAKFGAAGGLPGVGVPGVGVPGVGVPGVGVPGVGVPGVGVPGVGVPGVGVPGVGVPGVGVPGVGVPGALSPAATAKAAAKAAKFGARGAVGIGGIPTFGLGPGGFPGIGDAAAAPAAAAAKAAKIGAGGVGALGGVVPGAPGAIPGLPGVGGVPGVGIPAAAAAKAAAKAAQFGLGPGVGVAPGVGVVPGVGVVPGVGVAPGIGLGPGGVIGAGVPAAAKSAAKAAAKAQFRAAAGLPAGVPGLGVGAGVPGLGVGAGVPGLGVGAGVPGPGAVPGTLAAAKAAKFGPGGVGALGGVGDLGGAGIPGGVAGVVPAAAAAAKAAAKAAQFGLGGVGGLGVGGLGAVPGAVGLGGVSPAAAAKAAKFGAAGLGGVLGAGQPFPIGGGAGGLGVGGKPPKPFGGALGALGFPGGACLGKSCGRKRK

SEQ ID NO: 48 - protein, 733 aa, Bos taurus
MRSLTAAARRPEVLLLLLCILQPSQPGGVPGAVPGGVPGGVFFPGAGLGGLGVGGLGPGVKPAKPGVGGLVGPGLGAEGSALPGAFPGGFFGAGGGAAGAAAAYKAAAKAGAAGLGVGGIGGVGGLGVSTGAVVPQLGAGVGAGVKPGKVPGVGLPGVYPGGVLPGAGARFPGIGVLPGVPTGAGVKPKAQVGAGAFAGIPGVGPFGGQQPGLPLGYPIKAPKLPGFGPGGVAGSAGKAGYPTGTGVGPQAAAAAAKAAAKLGAGGAGVLPGVGVGGPGIPGAPGAIPGIGGIAGVGAPDAAAAAAAAAKAAKFGAAGGLPGVGVPGVGVPGVGVPGVGVPGVGVPGVGVPGVGVPGVGVPGVGVPGVGVPGVGVPGALSPAATAKAAAKAAKFGARGAVGIGGIPTFGLGPGGFPGIGDAAAAPAAAAAKAAKIGAGGVGALGGVVPGAPGAIPGLPGVGGVPGVGIPAAAAAKAAAKAAQFGLGPGVGVAPGVGVVPGVGVVPGVGVAPGIGLGPGGVIGAGVPAAAKSAAKAAAKAQFRAAAGLPAGVPGLGVGAGVPGLGVGAGVPGLGVGAGVPGPGAVPGTLAAAKAAKFGPGGVGALGGVGDLGGAGIPGGVAGVVPAAAAAAKAAAKAAQFGLGGVGGLGVGGLGAVPGAVGLGGVSPAAAAKAAKFGAAGLGGVLGAGQPFPIGGGAGGLGVGGKPPKPFGGALGALGFPGGACLGKSCGRKRK

SEQ ID NO: 49 - protein, 713 aa, Bos taurus
MRSLTAAARRPEVLLLLLCILQPSQPGGVPGAVPGGVPGGVFFPGAGLGGLGVGGLGPGVKPAKPGVGGLVGPGLGAEGSALPGAFPGGFFGAGGGAAGAAAAYKAAAKAGAAGLGVGGIGGVGGLGVSTGAVVPQLGAGVGAGVKPGKVPGVGLPGVYPGGVLPGAGARFPGIGVLPGVPTGAGVKPKAQVGAGAFAGIPGVGPFGGQQPGLPLGYPIKAPKLPGVGPQAAAAAAKAAAKLGAGGAGVLPGVGVGGPGIPGAPGAIPGIGGIAGVGAPDAAAAAAAAAKAAKFGAAGGLPGVGVPGVGVPGVGVPGVGVPGVGVPGVGVPGVGVPGVGVPGVGVPGVGVPGVGVPGALSPAATAKAAAKAAKFGARGAVGIGGIPTFGLGPGGFPGIGDAAAAPAAAAAKAAKIGAGGVGALGGVVPGAPGAIPGLPGVGGVPGVGIPAAAAAKAAAKAAQFGLGPGVGVAPGVGVVPGVGVVPGVGVAPGIGLGPGGVIGAGVPAAAKSAAKAAAKAQFRAAAGLPAGVPGLGVGAGVPGLGVGAGVPGLGVGAGVPGPGAVPGTLAAAKAAKFGPGGVGALGGVGDLGGAGIPGGVAGVVPAAAAAAKAAAKAAQFGLGGVGGLGVGGLGAVPGAVGLGGVSPAAAAKAAKFGAAGLGGVLGAGQPFPIGGGAGGLGVGGKPPKPFGGALGALGFPGGACLGKSCGRKRK

SEQ ID NO: 50 - protein, 860 aa, Mus musculus
MAGLTAVVPQPGVLLILLLNLLHPAQPGGVPGAVPGGLPGGVPGGVYYPGAGIGGLGGGGGALGPGGKPPKPGAGLLGTFGAGPGGLGGAGPGAGLGAFPAGTFPGAGALVPGGAAGAAAAYKAAAKAGAGLGGVGGVPGGVGVGGVPGGVGVGGVPGGVGVGGVPGGVGGIGGIGGLGVSTGAVVPQVGAGIGAGGKPGKVPGVGLPGVYPGGVLPGTGARFPGVGVLPGVPTGTGVKAKAPGGGGAFAGIPGVGPFGGQQPGVPLGYPIKAPKLPGGYGLPYTNGKLPYGVAGAGGKAGYPTGTGVGSQAAAAAAKAAKYGAGGAGVLPGVGGGGIPGGAGAIPGIGGIAGAGTPAAAAAAKAAAKAAKYGAAGGLVPGGPGVRLPGAGIPGVGGIPGVGGIPGVGGPGIGGPGIVGGPGAVSPAAAAKAAAKAAKYGARGGVGIPTYGVGAGGFPGYGVGAGAGLGGASPAAAAAAAKAAKYGAGGAGALGGLVPGAVPGALPGAVPAVPGAGGVPGAGTPAAAAAAAAAKAAAKAGLGPGVGGVPGGVGVGGIPGGVGVGGVPGGVGPGGVTGIGAGPGGLGGAGSPAAAKSAAKAAAKAQYRAAAGLGAGVPGFGAGAGVPGFGAGAGVPGFGAGAGVPGFGAGAGVPGFGAGAVPGSLAASKAAKYGAAGGLGGPGGLGGPGGLGGPGGLGGAGVPGRVAGAAPPAAAAAAAKAAAKAAQYGLGGAGGLGAGGLGAGGLGAGGLGAGGLGAGGLGAGGLGAGGLGAGGGVSPAAAAKAAKYGAAGLGGVLGARPFPGGGVAARPGFGLSPIYPGGGAGGLGVGGKPPKPYGGALGALGYQGGGCFGKSCGRKRK

SEQ ID NO: 51 - protein, 466 aa, Mus musculus
MAGLTAVVPQPGVLLILLLNLLHPAQPGGVPGAVPGGLPGGVPGGVYYPGAGIGGLGGGGGALGPGGKPPKPGAGLLGTFGAGPGGLGGAGPGAGLGAFPAGTFPGAGALVPGGAAGAAAAYKAAAKAGAGLGGVGGVPGGVGVGGVPGGVGVGGVPGGVGVGGVPGGVGGIGGIGGLGVSTGAVVPQVGAGIGAGGKPGKVPGVGLPGVYPGGVLPGTGARFPGVGVLPGVPTGTGVKAKAPGGGGAFAGIPGVGPFGGQQPGVPLGYPIKAPKLPGGYGLPYTNGKLPYGVAGAGGKAGYPTGTGVGSQAAAAAAKAAKYGAGGAGVLPGVGGGGIPGGAGAIPGIGGIAGAGTPAAAAAAKAAAKAAKYGAAGGLVPGGPGVRLPGAGIPGVGGIPGVGGIPGVGGPGIGGPGIVGGPGAVSPAAAAKAAAKAAKYGARGGVATHPPTHPSIHPSIHPSILTA

SEQ ID NO: 52 - protein, 375 aa, Mus musculus
ESRQVLWCPKSELASELEESLGKFLVLVFQVYTQAECSQEQELGSLVWGCSLEFPLAQESKPRLQVFGVGGIPGGVGVGGVPGGVGPGGVTGIGAGPGGLGGAGSPAAAKSAAKAAAKAQYRAAAGLGAGVPGFGAGAGVPGFGAGAGVPGFGAGAGVPGFGAGAGVPGFGAGAVPGSLAASKAAKYGAAGGLGGPGGLGGPGGLGGPGGLGGAGVPGRVAGAAPPAAAAAAAKAAAKAAQYGLGGAGGLGAGGLGAGGLGAGGLGAGGLGAGGLGAGGLGAGGLGAGGGVSPAAAAKAAKYGAAGLGGVLGARPFPGGGVAARPGFGLSPIYPGGGAGGLGVGGKPPKPYGGALGALGYQGGGCFGKSCGRKRK

SEQ ID NO: 53 - protein, 683 aa, Rattus norvegicus
MAGLTAAVPQPGVLLILLLNLLHPAQPGGVPGAVPGGVPGGLPGGVPGGVYYPGAGIGGGLGGGALGPGGKPPKPGAGLLGAFGAGPGGLGGAGPGAGLSYASRPGGVLVPGGGAGAAAAYKAAAKAGAGLGGIGGVPGGVGVGGVPGAVGVGGVPGAVGGIGGIGGLGVSTGAVVPQLGAGVGAGGKPGKVPGVGLPGVYPGGVLPGTGARFPGVGVLPGVPTGTGVKAKVPGGGGGAFSGIPGVGPFGGQQPGVPLGYPIKAPKLPGGYGLPYTNGKLPYGVAGAGGKAGYPTGTGVGSQAAVAAAKAAKYGAGGGGVLPGVGGGGIPGGAGAIPGIGGITGAGTPAAAAAAKAAAKAAKYGAVSPAAAAKAAAKAAKYGARGGVGIPTYGVGAGGFPGYGVGAGAGLGGASQAAAAAAAAKAAKYGAGGAGTLGGLVPGAVPGALPGAVPGALPGAVPGALPGAVPGVPGTGGVPGAGTPAAAAAAAAAKAAAKAGQYGLGPGVGGVPGGVGVGGLPGGVGPGGVTGIGTGPGTGLVPGDLGGAGTPAAAKSAAKAAAKAQYIPGSLAASKAAKYGGAPAAAAAAKAAAKAAQYGVSPAAAAKAAKYGAAGLGGVLGARPFPGGGVAARPGFGLSPIYPGGGAGGLGVGGKPPKPYGGALGALGYQGGGCFGKSCGRKRK

SEQ ID NO: 54 - protein, 633 aa, Rattus norvegicus
MAGLTAAVPQPGVLLILLLNLLHPAQPGGVPGAVPGGVPGGLPGGVPGGVYYPGAGIGGGLGGGALGPGGKPPKPGAGLLGAFGAGPGGLGGAGPGAGLSYASRPGGVLVPGGGAGAAAAYKAAAKAGAGLGGIGGVPGGVGVGGVPGAVGVGGVPGAVGGIGGIGGLGVSTGAVVPQLGAGVGAGGKPGKVPGVGLPGVYPGGVLPGTGARFPGVGVLPGVPTGTGVKAKVPGGGGGAFSGIPGVGPFGGQQPGVPLGYPIKAPKLPGGYGLPYTNGKLPYGVAGAGGKAGYPTGTGVGSQAAVAAAKAAKYGAGGGGVLPGVGGGGIPGGAGAIPGIGGITGAGTPAAAAAAKAAAKAAKYGAVSPAAAAKAAAKAAKYGARGGVGIPTYGVGAGGFPGYGVGAGAGLGGASQAAAAAAAAKAAKYGAGTPAAAAAAAAAKAAAKAGQYGLGPGVGGVPGGVGVGGLPGGVGPGGVTGIGTGPGTGLVPGDLGGAGTPAAAKSAAKAAAKAQYIPGSLAASKAAKYGGAPAAAAAAKAAAKAAQYGVSPAAAAKAAKYGAAGLGGVLGARPFPGGGVAARPGFGLSPIYPGGGAGGLGVGGKPPKPYGGALGALGYQGGGCFGKSCGRKRK

SEQ ID NO: 55 - protein, 406 aa, Rattus norvegicus
MAGLTAAVPQPGVLLILLLNLLHPAQPGGVPGAVPGGVPGGLPGGVPGGVYYPGAGIGGGLGGGALGPGGKPPKPGAGLLGAFGAGPGGLGGAGPGAGLSYASRPGGVLVPGGGAGAAAAYKAAAKAGAGLGGIGGVPGGVGVGGVPGAVGVGGVPGAVGGIGGIGGLGVSTGAVVPQLGAGVGAGGKPGKVPGVGLPGVYPGGVLPGTGARFPGVGVLPGVPTGTGVKAKVPGGGGGAFSGIPGVGPFGGQQPGVPLGYPIKAPKLPGGYGLPYTNGKLPYGVAGAGGKAGYPTGTGVGSQAAVAAAKAAKYGAGGGGVLPGVGGGGIPGGAGAIPGIGGITGAGTPAAAAAAKAAAKAAKYGAVSPAAAAKAAAKAAKYGARATHPRTHPSFHPSSIHPSVRPS

SEQ ID NO: 56 - protein, 5 aa, Artificial Sequence
  Elastin-like polypeptide
VPGXG

SEQ ID NO: 57 - protein, 5 aa, Artificial Seqeunce
  Elastin-like polypeptide
KGGVG

SEQ ID NO: 58 - protein, 5 aa, Artificial Seqeunce
  Elastin-like polypeptide
LGGVG

SEQ ID NO: 59 - protein, 7 aa, Artificial Sequence
  Elastin-like polypeptide
LGAGGAG

SEQ ID NO: 60 - protein, 9 aa, Artificial Sequence
  Elastin-like polypeptide
LGAGGAGVL

SEQ ID NO: 61 - protein, 5 aa, Artificial Sequence
  Leader peptide sequence
SKGPG

SEQ ID NO: 62 - protein, 208 aa, Artificial Sequence
  Elastin-like polypeptide
MSKGPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGWP

SEQ ID NO: 63 - protein, 808 aa, Artificial Sequence
  Elastin-like polypeptide
MSKGPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGVGVPGIGVPGIGVPGEGVPGIGVPGWP

SEQ ID NO: 64 - protein, 547 aa, Artificial Sequence
  Elastin-like polypeptide
MRFPSIFTAVLFAASSALAAPVNTTTEDETAQIPAEAVIGYSDLEGDFDVAVLPFSNSTNNGLLFINTTIASIAAKEEGVSLEKREAEAGPVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGA

SEQ ID NO: 65 - protein, 5263 aa, Bombyx mori
MRVKTFVILCCALQYVAYTNANINDFDEDYFGSDVTVQSSNTTDEIIRDASGAVIEEQITTKKMQRKNKNHGILGKNEKMIKTFVITTDSDGNESIVEEDVLMKTLSDGTVAQSYVAADAGAYSQSGPYVSNSGYSTHQGYTSDFSTSAAVGAGAGAGAAAGSGAGAGAGYGAASGAGAGAGAGAGAGYGTGAGAGAGAGYGAGAGAGAGAGYGAGAGAGAGAGYGAGAGAGAGAGYGAGAGAGAGAGYGAGAGAGAGAGYGAASGAGAGAGYGQGVGSGAASGAGAGAGAGSAAGSGAGAGAGTGAGAGYGAGAGAGAGAGYGAASGTGAGYGAGAGAGYGGASGAGAGAGAGAGAGAGAGYGTGAGYGAGAGAGAGAGAGAGYGAGAGAGYGAGYGVGAGAGYGAGYGAGAGSGAASGAGSGAGAGSGAGAGSGAGAGSGAGAGSGAGAGSGAGAGSGAGAGSGAGAGSGTGAGSGAGAGYGAGAGAGYGAGAGSGAASGAGAGSGAGAGSGAGAGSGAGAGSGAGAGSGAGAGYGAGAGAGYGAGAGAGYGAGAGVGYGAGAGSGAASGAGAGSGAGAGSGAGAGSGAGAGSGAGAGSGAGAGSGAGAGSGAGAGSGAGAGSGAGVGYGAGVGAGYGAGYGAGAGAGYGAGAGSGAASGAGAGAGAGAGTGSSGFGPYVANGGYSRSDGYEYAWSSDFGTGSGAGAGSGAGAGSGAGAGSGAGAGSGAGAGSGAGAGYGAGVGVGYGAGYGAGAGAGYGAGAGSGAASGAGAGSGAGAGSGAGAGSGAGAGSGAGAGSGAGAGSGAGAGSGAGAGSGAGAGSGAGAGSGAGVGSGAGAGSGAGAGVGYGAGAGVGYGAGAGSGAASGAGAGSGAGAGSGAGAGSGAGAGSGAGAGSGAGAGSGAGAGSGAGAGSGAGAGSGAGVGYGAGVGAGYGAGYGAGAGAGYGAGAGSGAASGAGAGSGAGAGSGAGAGSGAGAGSGAGAGSGAGAGSGAGAGSGAGAGSGAGAGSGAGAGSGAGAGSGAGAGYGAGAGAGYGAGYGAGAGAGYGAGAGSGAASGAGSGAGAGSGAGAGAGSGAGAGSGAGAGSGAGAGSGAGAGSGAGAGSGAGAGYGAGVGAGYGAGYGAGAGAGYGAGAGSGAASGAGAGSGAGAGSGAGAGSGAGAGSGAGAGSGAGAGSGAGAGSGAGVGYGAGYGAGAGAGYGAGAGSGAASGAGAGAGAGAGTGSSGFGPYVAHGGYSGYEYAWSSESDFGTGSGAGAGSGAGAGSGAGAGSGAGAGSGAGYGAGVGAGYGAGYGAGAGAGYGAGAGSGAGSGAGAGSGAGAGSGAGAGSGAGAGSGAGAGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGAGYGAGVGAGYGAGYGAGAGAGYGAGAGSGAGSGAGAGSGAGAGSGAGAGSGAGVGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGVGYGAGVGAGYGAGYGAGAGAGYGAGAGSGAASGAGAGAGAGAGTGSSGFGPYVANGGYSGYEYAWSSESDFGTGSGAGAGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGSGSGAGAGSGAGAGSGAGAGYGAGVGAGYGVGYGAGAGAGYGAGAGSGAASGAGAGAGAGAGTGSSGFGPYVAHGGYSGYEYAWSSESDFGTGSGAGAGSGAGAGSGAGAGSGAGAGSGAGAGSGAGAGSGAGAGYGAGVGAGYGAAYGAGAGAGYGAGAGSGAASGAGAGSGAGAGSGAGAGSGAGAGSGAGAGSGAGAGSGAGAGSGAGAGSGAGAGSGAGAGSGAGAGYGAGAGAGYGAGAGSGAGSGAGAGSGAGAGSGAGAGSGAGAGSGAGAGSGAGSGSGAGAGSGAGAGSGAGAGYGAGVGAGYGAGYGAGAGAGYGAGAGSGAGSGAGAGSGAGAGYGAGAGAGYGAGYGAGAGAGYGAGAGTGAGSGAGAGSGAGAGSGAGAGSGAGAGSGAGAGSGAGAGSGAGSGSGAGAGSGAGAGSGAGAGSGAGAGSGAGAGSGAGAGYGAGAGAGYGAGYGAGAGAGYGAGAGSGAGSGAGAGSGAGAGSGAGAGSGAGAGYGAGYGAGAGSGAASGAGAGAGAGAGTGSSGFGPYVAHGGYSGYEYAWSSESDFGTGSGAGAGSGAGAGAGAGAGSGAGAGYGAGVGAGYGAGYGAGAGAGYGAGAGSGTGSGAGAGSGAGAGYGAGVGAGYGAGAGSGAAFGAGAGAGAGSGAGAGSGAGAGSGAGAGSGAGAGSGAGAGYGAGYGAGVGAGYGAGAGSGAASGAGAGSGAGAGSGAGAGSGAGAGSGAGAGSGAGAGYGAGVGAGYGAGYGAGAGAGYGAGAGSGAASGAGAGSGAGAGAGSGAGAGSGAGAGSGAGAGSGAGSGAGAGSGAGAGSGAGAGYGAGAGSGAASGAGAGAGAGAGTGSSGFGPYVANGGYSGYEYAWSSESDFGTGSGAGAGSGAGAGSGAGAGSGAGAGSGAGAGYGAGVGAGYGAGYGAGAGAGYGAGAGSGAGSGAGAGSGAGAGSGAGAGSGAGAGSGAGAGSGAGAGSGAGAGYGAGAGSGAASGAGAGSGAGAGSGAGAGSGAGAGSGAGAGSGAGAGSGAGAGYGAGVGAGYGVGYGAGAGAGYGAGAGSGAGSGAGAGSGAGAGSGAGAGSGAGAGSGAGSGAGAGSGAGAGSGAGAGSGAGSGAGAGSGAGAGYGVGYGAGAGAGYGAGAGSGAGSGAGAGSGAGAGSGAGAGSGAGSGAGAGSGAGAGSGAGAGSGAGAGYGAGVGAGYGVGYGAGAGAGYGAGAGSGAGSGAGAGSGAGAGSGAGAGSGAGAGSGAGAGSGAGAGSGAGAGSGAGSGAGAGSGAGAGSGAGAGSGAGAGSGAGSGAGAGSGAGAGSGAGAGSGAGAGYGAGVGAGYGVGYGAGVGAGYGAGAGSGAASGAGAGSGAGAGAGSGAGAGSGAGAGSGAGAGSGAGAGSGAGAGSGAGAGYGAGYGAGVGAGYGAGAGVGYGAGAGAGYGAGAGSGAASGAGAGAGSGAGAGTGAGAGSGAGAGYGAGAGSGAASGAGAGAGAGAGTGSSGFGPYVANGGYSGYEYAWSSESDFGTGSGAGAGSGAGAGSGAGAGSGAGAGSGAGAGYGAGVGAGYGAGAGSGAGSGAGAGSGAGAGSGAGAGSGAGAGSGAGAGYGAGAGSGTGSGAGAGSGAGAGSGAGAGSGAGAGSGAGAGSGAGAGSGVGAGYGVGYGAGAGAGYGVGYGAGAGAGYGAGAGSGTGSGAGAGSGAGAGSGAGAGSGAGAGSGAGAGSGAGAGSGAGAGYGAGVGAGYGVGYGAGAGAGYGAGAGSGAGSGAGAGSGAGAGSGAGAGSGAGAGSGAGSGAGAGSGAGAGSGAGAGSGAGSGAGAGSGAGAGYGVGYGAGAGAGYGAGAGSGAGSGAGAGSGAGAGSGAGAGSGAGSGAGAGSGAGAGSGAGAGSGAGAGYGAGVGAGYGVGYGAGAGAGYGAGAGSGAGSGAGAGSGAGAGSGAGAGSGAGAGSGAGAGSGAGAGSGAGAGSGAGSGAGAGSGAGAGSGAGAGSGAGAGYGAGVGAGYGVGYGAGAGAGYGAGAGSGAASGAGAGAGAGAGTGSSGFGPYVANGGYSGYEYAWSSESDFGTGSGAGAGSGAGAGSGAGAGYGAGYGAGVGAGYGAGAGVGYGAGAGAGYGAGAGSGAASGAGAGAGAGAGSGAGAGSGAGAGAGSGAGAGYGAGYGIGVGAGYGAGAGVGYGAGAGAGYGAGAGSGAASGAGAGSGAGAGSGAGAGSGAGAGSGAGAGSGAGAGSGAGAGYGAGYGAGVGAGYGAGAGVGYGAGAGAGYGAGAGSGAASGAGAGAGAGAGAGSGAGAGSGAGAGSGAGAGSGAGAGSGAGAGSGAGAGSGAGAGSGAGAGSGAGAGYGAGVGAGYGAGYGGAGAGYGAGAGSGAASGAGAGSGAGAGSGAGAGSGAGAGSGAGAGSGAGAGYGAGAGSGAASGAGAGAGAGAGTGSSGFGPYVNGGYSGYEYAWSSESDFGTGSGAGAGSGAGAGSGAGAGYGAGVGAGYGAGYGAGAGAGYGAGAGSGAASGAGAGSGAGAGSGAGAGSGAGAGSGAGSGAGAGSGAGAGSGAGAGSGAGAGSGAGAGSGAGAGYGAGVGAGYGAGYGAGAGAGYGAGAGSGAASGAGAGSGAGAGAGSGAGAGSGAGAGSGAGAGSGAGAGSGAGAGSGAGSGAGAGSGAGAGYGAGYGAGVGAGYGAGAGVGYGAGAGAGYGAGAGSGAASGAGAGSGSGAGSGAGAGSGAGAGSGAGAGAGSGAGAGSGAGAGSGAGAGYGAGYGAGAGSGAASGAGAGAGAGAGTGSSGFGPYVANGGYSGYEYAWSSESDFGTGSGAGAGSGAGAGSGAGAGYGAGVGAGYGAGYGAGAGAGYGAGAGSGAGSGAGAGSGAGAGSGAGAGSGAGAGSGAGAGSGAGAGSGAGAGSGAGAGYGAGYGAGAGAGYGAGAGVGYGAGAGAGYGAGAGSGAGSGAGAGSGSGAGAGSGSGAGSGAGAGSGAGAGSGAGAGSGAGAGSGAGAGSGAGAGSGAGAGYGAGYGIGVGAGYGAGAGVGYGAGAGAGYGAGAGSGAASGAGAGSGAGAGSGAGAGSGAGAGSGAGAGSGAGAGSGAGAGSGAGAGSGAGAGSGAGAGYGAGAGVGYGAGAGSGAASGAGAGSGAGAGSGAGAGSGAGAGSGAGAGSGAGAGSGAGAGSGAGSGAGAGSGAGAGYGAGYGAGVGAGYGAGAGYGAGYGVGAGAGYGAGAGSGAGSGAGAGSGAGAGSGAGAGSGAGAGSGAGAGSGAGSGAGAGYGAGAGAGYGAGAGAGYGAGAGSGAASGAGAGAGAGSGAGAGSGAGAGSGAGSGAGAGSGAGAGYGAGAGSGAASGAGAGSGAGAGAGAGAGAGSGAGAGSGAGAGYGAGAGSGAASGAGAGAGAGTGSSGFGPYVANGGYSRREGYEYAWSSKSDFETGSGAASGAGAGAGSGAGAGSGAGAGSGAGAGSGAGAGGSVSYGAGRGYGQGAGSAASSVSSASSRSYDYSRRNVRKNCGIPRRQLVVKFRALPCVNC

SEQ ID NO: 66 - protein, 262 aa, Bombyx mori; identical to SEQ ID NO: 2

SEQ ID NO: 67 - protein, 220 aa, Bombyx mori
MLARCLAVAAVAVLASAGPPSPIYRPCYLDDYKCISDHLAANSKCIPGRGQIPSQYEIPVFQFEIPYFNATYVDHNLITRNHDQCRVSEFYDNVRTLKTVLTVDCPWLNFESNRTLAQHMSFKEDVVLSFYINGSYPLIRLTTVFDKGNNFDLCSAFTFADLAGGLPIFHINPNDQRTAQWLSKDLTLLHIYEREHIFGKRNWLARSFISRTLCDFGCQH

SEQ ID NO: 68 - protein, 6 aa, Artificial Sequence
  Fibroin-like polypeptide
GAGAGS

SEQ ID NO: 69 - protein, 5 aa, Artificial Sequence
  Fibroin-like polypeptide
GAAGY

SEQ ID NO: 70 - protein, 9 aa, Artificial Sequence
  Fibroin-like polypeptide
AGAGAGPEG

SEQ ID NO: 71 - protein, 8 aa, Artificial Sequence
  Fibroin-like polypeptide
AGAGAGEG

SEQ ID NO: 72 - protein, 59 aa, Artificial Sequence
  Fibroin-like polypeptide
GAGAGSGAAGGAGAGSGAGAGSGAGAGSGAGAGSGAGAGSGAGAGSGAGAGSGAGAGSY

SEQ ID NO: 73 - protein, 13 aa, Artificial Sequence
  Fibroin-like polypeptide
YGGLGSQGAGRGG

SEQ ID NO: 74 - protein, 878 aa, Artificial Sequence
  Silk-elastin-like polypeptide (SELP)
MDPVVLQRRDWENPGVTQLNRLAAHPPFASDPMGAGSGAGAGSGVGVPGVGVPGVGVPGVGVPGKGVPGVGVPGVGVPGVGVPGAGAGSGAGAGSGAGAGSGAGAGSGVGVPGVGVPGVGVPGVGVPGKGVPGVGVPGVGVPGVGVPGAGAGSGAGAGSGAGAGSGAGAGSGVGVPGVGVPGVGVPGVGVPGKGVPGVGVPGVGVPGVGVPGAGAGSGAGAGSGAGAGSGAGAGSGVGVPGVGVPGVGVPGVGVPGKGVPGVGVPGVGVPGVGVPGAGAGSGAGAGSGAGAGSGAGAGSGVGVPGVGVPGVGVPGVGVPGKGVPGVGVPGVGVPGVGVPGAGAGSGAGAGSGAGAGSGAGAGSGVGVPGVGVPGVGVPGVGVPGKGVPGVGVPGVGVPGVGVPGAGAGSGAGAGSGAGAGSGAGAGSGVGVPGVGVPGVGVPGVGVPGKGVPGVGVPGVGVPGVGVPGAGAGSGAGAGSGAGAGSGAGAGSGVGVPGVGVPGVGVPGVGVPGKGVPGVGVPGVGVPGVGVPGAGAGSGAGAGSGAGAGSGAGAGSGVGVPGVGVPGVGVPGVGVPGKGVPGVGVPGVGVPGVGVPGAGAGSGAGAGSGAGAGSGAGAGSGVGVPGVGVPGVGVPGVGVPGKGVPGVGVPGVGVPGVGVPGAGAGSGAGAGSGAGAGSGAGAGSGVGVPGVGVPGVGVPGVGVPGKGVPGVGVPGVGVPGVGVPGAGAGSGAGAGSGAGAGSGAGAGSGVGVPGVGVPGVGVPGVGVPGKGVPGVGVPGVGVPGVGVPGAGAGSGAGAGSGAGAGSGAGAGSGVGVPGVGVPGVGVPGVGVPGKGVPGVGVPGVGVPGVGVPGAGAGSGAGAGSGAGAMDPGRYQDLRS

SEQ ID NO: 75 - protein, 670 aa, Artificial Sequence
  Silk-elastin-like polypeptide (SELP)
MDPVVLQRRDWENPGVTQLNRLAAHPPFASDPMGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAMDPGRYQDLRS

SEQ ID NO: 76 - protein, 878 aa, Artificial Sequence
  Silk-elastin-like polypeptide (SELP)
MDPVVLQRRDWENPGVTQLNRLAAHPPFASDPMGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAMDPGRYQDLRS

SEQ ID NO: 77 - protein, 1086 aa, Artificial Sequence
  Silk-elastin-like polypeptide (SELP)
MDPVVLQRRDWENPGVTQLNRLAAHPPFASDPMGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAGSGAGAGSGVGVPGVGVPGVGVPGVGVPGKGVPGVGVPGVGVPGVGVPGVGVPGVGVPGVGVPGVGVPGVGVPGVGVPGVGVPGVGVPGAGAGSGAGAGSGAGAMDPGRYQDLRS

SEQ ID NO: 78 - protein, 10 aa, Artificial Sequence
  Abductin-like polypeptide
GGFGGMGGGX

SEQ ID NO: 79 - protein, 136 aa, Argopecten irradians
MNAYICLAACLIAAVSAAGYGGGAGSMGGTGGMGGGMNAGGFGGMGGGMGGGKGGFGGIGGFGGMGGGMGGGPGGFGGMGGFGGMGGGKGGFGGMGSGMGGFGGMGGGNAGFGGMGGGNAGFGGMGGQGGFGGKGY

SEQ ID NO: 80 - protein, 132 aa, Argopecten irradians
MNAYICLAACLIAVVSAAGYGGGAGSMGGTGGMGGGMNAGGFGGIGGGMGGGKGGFGGMGGGPGGFGGIGGGSGGFGGMGGFGGMGGGKGGFGGMGSSMGGFGGMGGGNAGFGGMGGQSGMGGQSGFGGKGY

SEQ ID NO: 81 - protein, 131 aa, Argopecten irradians
MNAYICLAACLIAAVSAAGYGGGAGSMGGTGGMGGGMNAGGFGGMGGMGGGKGGFGGIGGFGGGMGGGPGGFGGMGGFGGMAAKGGFGGMGSGMGGFGGMGGGNAGFGGMGGGNAGFGGMGGQGGFGGKGY

SEQ ID NO: 82 - protein, 126 aa, Argopecten irradians
MNAYICLSACLIAAVSAAGYGGGAGSMGGTGGMGGGMNAGGFGGMGGGMGGGKGGFGGMGGFGGMGGGMGGGPGGFGGMGGFGGMGGGKGGFGGMGSGMGGFGGMGGGNAGFGGMGGQGGFGGKGY

SEQ ID NO: 83 - protein, 290 aa, Triticum aestivum
MVRVPVPQLQPQNPSQQQPQEQVPLVQQQQFPGQQQPFPPQQPYPQPQPFPSQQPYLQLQPFPQPQLPYPQPQLPYPQPQLPYPQPQPFRPQQPYPQSQPQYSQPQQPISQQQQQQQQQQQQKQQQQQQQQILQQILQQQLIPCRDVVLQQHSIAYGSSQVLQQSTYQLVQQLCCQQLWQIPEQSRCQAIHNVVHAIILHQQQQQQQQQQQQPLSQVSFQQPQQQYPSGQGSFQPSQQNPQAQGSVQPQQLPQFEEIRNLALETLPAMCNVYIPPYCTIAPVGIFGTNYR

SEQ ID NO: 84 - protein, 288 aa, Triticum aestivum
MKTFLILALLAIVATTATTAVRVPVPQLQPQNPSQQQPQEQVPLVQQQQFLGQQQPFPPQQPYPQPQPFPSQQPYLQLQPFPQPQLPYSQPQPFRPQQPYPQPQPQYSQPQQPISQQQQQQQQQQQQQQQQQQQQILQQILQQQLIPCMDVVLQQHNIAHGRSQVLQQSTYQLLQELCCQHLWQIPEQSQCQAIHKVVHAIILHQQQKQQQQPSSQVSFQQPLQQYPLGQGSFRPSQQNPQAQGSVQPQQLPQFEEIRNLALQTLPAMCNVYIPPYCTITPFGIFGTN

SEQ ID NO: 85 - protein, 278 aa, Triticum aestivum
MVRVPVPQLQPQNPSQQQPQEQVPLVQQQQFLGQQQPFPPQQPYPQPQPFPSQQPYLQLQPFPQPQLSYSQPQPFRPQQLYPQPQPQYSQPQQPISQQQQQQQQQQQQQQQQQQQQQQQQEQQILQQMLQQQLIPCMDVVLQQHNIAHGRSQVLQQSTYQLLQELCCQHLWQILEQSQCQAIHNVVHAIILHQQQKQQQQPSSQVSFQQPLQQYPLGQGSFRPSQQNPQAQGSVQPQQLPQFEEIRNLALQTLPAMCNVYIPPYCTIAPFGIFGTNYR

SEQ ID NO: 86 - protein, 277 aa, Triticum aestivum
MVRVPVPQLQPQNPSQQHPQEQVPLVQQQQFLGQQQSFPPQQPYPQPQPFPSQQPYLQLQPFPQPQLPYLQPQPFRPQQPYPQPQPQYSQPQQPISQQQQQQQQQQQQQQQQQQQQQQQQQQILQQILQQQLIPCMDVVLQQHNIAHGRSQVLQQSTYQLLQELCCQHLWQIPEQSQCQAIHNVVHAIILHQQQKQQQQPSSQVSFQQPLQQYPLGQGSFRPSQQNPLAQGSVQPQQLPQFEEIRNLALQTLPAMCNVYIPPYCTIVPFGIFGTNYR

SEQ ID NO: 87 - protein, 276 aa, Triticum aestivum
MVRVPVPQLQPQNPSQQQPQEQVPLVQQQQFLGQQQPFPPQQPYPQPQPFPSQQPYLQLQPFPQPQLSYSQPQPFRPQQPYPQPQPQYSQPQQPISQQQQQQQQQQQQQQQQQQQQQQEQQILQQILQQQLIPCMDVVLQQHNIAHGRSQVLQQSTYQLLQELCCQHLWQIPEQSQCQAIHNVVHAIILHQQQKQQQQPSSQVSFQQPLQQYPLGQGSFRPSQQNPQAQGSVQPQQLPQFEEIRNLALQTLPAMCNVYIPPYCTMAPFGIFGTNYR

SEQ ID NO: 88 - protein, 274 aa, Triticum aestivum
MVRVTVPQLQPQNPSQQQPQEQVPLVQQQQFLGQQQPFPPQQPYPQPQPFPSQQPYLQLQPFPQPQLPYSQPQPFRPQQPYPQPQPQYSQPQQPISQQQQQQQQQQQQQQQQQQQQQQQILQQILQQQLIPCMDVVLQQHNIVHGRSQVLQQSTYQLLQELCCQHLWQIPEQSQCQAIHNVVHAIILHQQQKQQQQPSSQVSFQQPLQQYPLGQGSFRPSQQNPQAQGSVQPQQLPQFEEIRNLALQTLPAMCNVYIPPYCTIAPFGIFGTNYR

SEQ ID NO: 89 - protein, 273 aa, Triticum aestivum
MVRVPVPQLQLQNPSQQQPQEQVPLVQEQQFPGQQQPFPPQQPYPQPQPFPSQQPYLQLQPFPRPQLPYPQPQPFRPQQPYPQPQPQYSQPQQPISQQQQQQQQQQQQQQILQQILQQQLIPCRDVVLQQHNIAHGSSQVLQESTYQLVQQLCCQQLWQIPEQSRCQAIHNVVHAIILHQQHHHHQQQQQQQQQQPLSQVSFQQPQQQYPSGQGFFQPFQQNPQAQGSFQPQQLPQFEAIRNLALQTLPAMCNVYIPPYCTIAPFGIFGTNYR

SEQ ID NO: 90 - protein, 270 aa, Triticum aestivum
MVRVPMPQLQPQDPSQQQPQEQVPLVQQQQFLGQQQPFPPQQPYPQPQPFPSQQPYLQLQPFPQPQLPYSQPQPFRPQQPYPQPQPQYSQPQQPISQQQQQQQQQQQQQQQQEQQILQQILQQQLIPCMDVVLQQHNLAHGRSQVLQQSTYQLLQELCCQHLWQIPEQSQCQAIHNVVHAIILHQQQKQQQQLSSQVSFQQPQQQYPLGQGSFRPSQQNSQAQGSVQPQQLPQFEEIRNLALQTLPAMCNVYIPPYCTIAPFGIFGTNYR

SEQ ID NO: 91 - protein, 269 aa, Triticum aestivum
MVRVPVPQLQPQNPSQQQPQEQVPLVQQQQFLGQQQPFPPQQPYPQPQPFPSQLPYLQLQPFPQPQLPYSQPQPFRPQQPYPQPQPQYSQPQQPISQQQQQQQQQQQQQQQQQQILQQILQQQLIPCMDVVLQQHNIAHGRSQVLQQSTYQLLQELCCQHLWQIPEQSQCQAIHNVVHAIILHQQQKQQQQPSSQVSFQQPLQQYPLGQGSFRPSQQNPQAQGSVQPQQLPQFEEIRNLALQTLPAMCNVYIPPYCTIAPFGIFGTNYR

SEQ ID NO: 92 - protein, 265 aa, Triticum aestivum
MVRVPVPQLQPQNPSQQQPQEQVPLMQQQQQFPGQQEQFPPQQPYPHQQPFPSQQPYPQPQPFPPQLPYPQTQPFPPQQPYPQPQPQYPQPQQPISQQQAQQQQQQQQILQQILQQQLIPCRDVVLQQHNIAHASSQVLQQSSYQQLQQLCCQQLFQIPEQSRCQAIHNVVHAIILHHHQQQQQQPSSQVSYQQPQEQYPSGQGSFQSSQQNPQAQGSVQPQQLPQFQEIRNLALQTLPAMCNVYIPPYCSTTIAPFGIFGTNYR

SEQ ID NO: 93 - protein, 337 aa, Triticum aestivum
MKTLLILTIIAVALTTTTANIQVDPSGQVQWPQQQQPFPQPQQPFSQQPQQIFPQPQQTFPHQPQQAFPQPQQTFPHQPQQQFPQPQQPQQPFPQQPQQQFPQPQQPQQPFPQQPQQQFPQPQQPQQPFPQPQQPQLPFPQQPQQPFPQPQQPQQPFPQLQQPQQPLPQPQQPQQPFPQQQQPLIQPYLQQQMNPCKNYLLQQCNPVSLVSSLVSMILPRSDCKVMRQQCCQQLARIPQQLQCAAIHGIVHSIIMQQEQQQQQQQQQQQQQGIQIMRPLFQLVQGQGIIQPQQPAQLEVIRSLVLGTLPTMCNVFVPPECSTTKAPFASIVADIGGQ

SEQ ID NO: 94 - protein, 327 aa, Triticum aestivum
MKTLLILTILAMAITIGTANIQVDPSGQVQWLQQQLVPQLQQPLSQQPQQTFPQPQQTFPHQPQQQVPQPQQPQQPFLQPQQPFPQQPQQPFPQTQQPQQPFPQQPQQPFPQTQQPQQPFPQQPQQPFPQTQQPQQPFPQLQQPQQPFPQPQQQLPQPQQPQQSFPQQQRPFIQPSLQQQLNPCKNILLQQSKPASLVSSLWSIIWPQSDCQVMRQQCCQQLAQIPQQLQCAAIHSVVHSIIMQQQQQQQQQQGIDIFLPLSQHEQVGQGSLVQGQGIIQPQQPAQLEAIRSLVLQTLPSMCNVYVPPECSIMRAPFASIVAGIGGQ

SEQ ID NO: 95 - protein, 314 aa, Triticum aestivum
MKTLLILTILAMATTIATANMQVDPSGQVQWPQQQPFPQPQQPFCEQPQRTIPQPHQTFHHQPQQTFPQPEQTYPHQPQQQFPQTQQPQQPFPQPQQTFPQQPQLPFPQQPQQPFPQPQQPQQPFPQSQQPQQPFPQPQQQFPQPQQPQQSFPQQQQPLIQPYLQQQMNPCKNYLLQQCNPVSLVSSLVSMILPRSDCKVMRQQCCQQLAQIPQQLQCAAIHGIVHSIIMQQEQQQQQQQQQQQQQQQGIQIMRPLFQLVQGQGIIQPQQPAQLEVIRSLVLGTLPTMCNVFVPPECSTTKAPFASIVADIGGQ

SEQ ID NO: 96 - protein, 308 aa, Triticum aestivum
MKTLLIQTILVMAITIATANMQVDPSGQVPRPQQQPFPQPHQPFSQQPQQTFPQPQQTFPHQPQQQFSQPQQPQQQFIQPQQPFPQQPQQTYPQRPQQPFPQTQQPQQPFPQSQQPQQPFPQSQQPQQPFPQPQQQFPQPQQPQQSFPQQQPSLIQQSLQQQLNPCKNFLLQQCKPVSLVSSLWSMILPRSDCQVMRQQCCQQLAQIPQQLQCAAIHSIVHSIIMQQEQQEQRQGVQILVPLSQQQQVGQGTLVQGQGIIQPQQPAQLEVIRSLVLQTLATMCNVYVPPYCSTIRAPFASIVAGIGGQ

SEQ ID NO: 97 - protein, 302 aa, Triticum aestivum
MKTLLILTILAMATTIATANMQVDPSGQVQWPQQQPFPQPQQPFCEQPQRTIPQPHQTFHHQPQQTFPQPEQTYPHQPQQQFPQTQQPQQPFPQPQQTFPQQPQLPFPQQPQQPFPQPQQPQQPFPQSQQPQQPFPQPQQQFPQPQQPQQSFPQQQQPAIQSFLQQQMNPCKNFLLQQCNHVSLVSSLVSIILPRSDCQVMQQQCCQQLAQIPQQLQCAAIHSVAHSIIMQQEQQQGVPILRPLFQLAQGLGIIQPQQPAQLEGIRSLVLKTLPTMCNVYVPPDCSTINVPYANIDAGIGGQ

SEQ ID NO: 98 - protein, 298 aa, Triticum aestivum
MKTLLILTILAMAVTIGTANMQVGPSGQVQWPQQQPVLLPQQPFSQQPQQTFPQPQQTFPHQPQQQFSQPQQPQQQFIQPQQPFPQQPQQTYPQRPQQPFPQTQQPQQPFPQSQQPQQPFPQPQQQFPQPQQPQQSFPQQQPSLIQQSLQQQLNPCKNFLLQQCKPVSLVSSLWSMILPRSDCQVMRQQCCQQLAQIPQQLQCAAIHSIVHSIIMQQEQQEQRQGVQILVPLSQQQQVGQGTLVQGQGIIQPQQPAQLEVIRSSVLQTLATMCNVYVPPYCSTIRAPFASIVAGIGGQ

SEQ ID NO: 99 - protein, 295 aa, Triticum aestivum
MKTLLIVTILAMATTIATANMQVDPGYQVQWPQQQPSPQPQQPFCQQPQQTIPQPHQTFHHQPQQTYPHQPQQQFPQTQQPQQPFPQPQQTFPQQPQLPFPQQPQQPFPQPQQPQQQFPQSQQPQQPLPQPQQQFLQPQQPQQSFPQQQQPLIQLSLQQQMNPCKNFLLQQCNPVSLVSSLISMILPRSDCQVMQQQCCQQLAQIPQQLQCAAIHSVVHSIIMQQEQRQGVQIRRPLFQLVQGQGIIQPQQPAQLEVIRSLVLRTLPTMCNVYVSPDCSTINAPFANIVVGIGGQ

SEQ ID NO: 100 - protein, 282 aa, Triticum aestivum
MNIQVDPSSQVPWPQQQPFPQPHQPFSQQPQQTFPQPQQTFPHQPQQQFSQPQQPQQQFIQPQQPFPQQPQQTYPQRPQQPFPQTQQPQQPFPQSQQPQQPFPQPQQQFPQPQQPQQSFPQQQPSLIQQSLQQQLNPCKNFLLQQCKPVSLVSSLWSMILPRSDCQVMRQQCCQQLAQIPQQLQCAAIHSIVHSIIMQQEQQEQRQGVQILVPLSQQQQVGQGTLVQGQGIIQPQQPAQLEVIRSLVLQTLATMCNVYVPPYCSTIRAPFASIVAGIGGQYR

SEQ ID NO: 101 - protein, 279 aa, Triticum aestivum
MNIQVDPSSQVQWPQQQPVPQPHQPFSQQPQQTFPQPQQTFPHQPQQQFPQPQQPQQQFLQPQQPFPQQPQQPYPQQPQQPFPQTQQPQQLFPQSQQPQQQFSQPQQQFPQPQQPQQSFPQQQPPFIQPSLQQQVNPCKNFLLQQCKPVSLVSSLWSMIWPQSDCQVMRQQSCQQLAQIPQQLQCAAIHTVIHSIIMQQEQQQGMHILLPLYQQQQVGQGTLVQGQGIIQPQQPAQLEAIRSLVLQTLPTMCNVYVPPECSIIKAPFSSVVAGIGGQYR

SEQ ID NO: 102 - protein, 277 aa, Triticum aestivum
MNIQVDPSGQVPWPQQQPFPQPHQPFSQQPQQTFPQPQQTFPHQPQQQFSQPQQPQQQFIQPQQPQQTYPQRPQQPFPQTQQPQQPFPQSQQPQQPFPQPQQQFPQPQQPQQSFPQQQPSLIQQSLQQQLNPCKNFLLQQCKPVSLVSSLWSMILPRSDCQVMRQQCCQQLAQIPQQLQCAAIHSIVHSIIMQQEQQEQRQGVQILVPLSQQQQVGQGTLVQGQGIIQPQQPAQLEVIRSLVLQTLATMCNVYVPPYCSTIRAPFASIVAGIGGQYR

SEQ ID NO: 103 - protein, 275 aa, Triticum aestivum
MNIQVDPSSQVQWPQQQPFLQPHQPFSQQPQQIFPQPQQTFPHQPQQQFPQPQQPQQQFLQPRQPFPQQPQQPYPQQPQQPFPQTQQPQQPFPQSKQPQQPFPQPQQPQQSFPQQQPSLIQQSLQQQLNPCKNFLLQQCKPVSLVSSLWSIILPPSDCQVMRQQCCQQLAQIPQQLQCAAIHSVVHSIIMQQEQQEQLQGVQILVPLSQQQQVGQGILVQGQGIIQPQQPTQLEVIRSLVLQTLPTMCNVYVPPYCSTFRAPFASIVAGIGGQYR

SEQ ID NO: 104 - protein, 274 aa, Triticum aestivum
MNIQVDPSSQVQWPQQQPFLQPHQPFSQQPQQIFPQPQQTFPHQPQQQFSQPQQPQQQFIQPQQPFPQQPQQPFPQTQQPQQPFPQSQQPQQPFPQPQQQFPQPQQPQQSFPQQQPSLIQQSLQQQLNPCKNFLLQQCKPVSLVSSLWSMILPRSDCQVMRQQCCQQLAQIPQQLQCAAIHSIVHSIIMQQEQQEQRQGVQILVPLSQQQQVGQGILVQGQGIIQPQQPTQLEVIRSLVLQTLPTMCNVYVPPKCSIMRAPFASIVAGIGGQYR

SEQ ID NO: 105 - protein, 258 aa, Triticum aestivum
MKTLLILTILAMAITIGTANIQVDPSGQVQWLQQQLVPQLQQPLSQQPQQTFPQPQQTFPQTQQPQQPFPQLQQPQQPFPQPQQQLPQPQQPQQSFPQQQRSFIQPSLQQQLNPCKNILLQQCKPASLVSSLWSIIWPQSDCQVMRQQCCQQLAQIPQQLQCAAIHSVVHSIIMQQQQQQQQQQGMHIFLPLSQQQQVGQGSLVQGQGIIQPQQPAQLEAIRSLVLQTLPSMCNVYVPPECSIMRAPFASIVAGIGGQ

SEQ ID NO: 106 - protein, 257 aa, Triticum aestivum
MNIQVDPSSQVQWPQQQPVPQPHQPFSQQPQQQFLQPQQPFPQQPQQPYPQQPQQPFPQTQQPQQLFPQSQQPQQQFSQPQQQFPQPQQPQQSFPQQQPPFIQPSLQQQVNPCKNFLLQQCKPVSLVSSLWSMIWPQSDCQVMRQQCCQQLAQIPQQLQCAAIHTIIHSIIMQQEQQEQQQGMHILLPLYQQQQVGQGTLVQGQGIIQPQQPAQLEAIRSLVLQTLPTMCNVYVPPECSIIKAPFSSVVAGIGGQYR

SEQ ID NO: 107 - protein, 256 aa, Triticum aestivum
MKTLLILTIIAVALTTTTANIQVDPSGQVQWPQQQQPFPQPQQPQQPFPQPQQPQLPFPQQPQQPFPQPQQPQQPFPQLQQPQQPLPQPQQPQQPFPQQQQPLIQPYLQQQMNPCKNYLLQQCNPVSLVSSLVSMILPRSDCKVMRQQCCQQLARIPQQLQCAAIHGIVHSIIMQQEQQQQQQQQQQQQQGIQIMRPLFQLVQGQGIIQPQQPAQLEVIRSLVLGTLPTMCNVFVPPECSTTKAPFASIVADIGGQ

SEQ ID NO: 108 - protein, 251 aa, Triticum aestivum
MKTLLILTILAMAITIGTANMQVDPSSQVQWPQQQPVPQPHQPFSQQPQQTFPQPQQTFPHQPQQQFPQPQQPQQQFLQPQQPFPQQPQQPYPQQPQQPFPQTQQPQQLFPQSQQPQQQFSQPQQQFPQPQQPQQSFPQQQPPFIQPSLQQQVNPCKNFLLQQCKPVSLVSSLWSMIWPQSDCQVMRQQCCQQLAQIPQQLQCAAIHTIIHSIIMQQEQQEQQQGMHILLPLYQQQQVGQGTLVQGQGIIQ

SEQ ID NO: 109 - protein, 241 aa, Triticum aestivum
MKTLLILTIIAVALTTTTANIQVDPSGQVQWPQQQQPFPQPQQPFSQQPQQIFPQPQQTFPHQPQQAFPQPQQTFPHQPQQQFPQPQQPQQPFPQQPQQQFPQPQQPQQPFPQQPQQQFPQPQQPQQPFPQPQQPQLPFPQQPQQPFPQPQQPQQPFPQLQQPQQPLPQPQQPQQPFPQQQQPLIQPYLQQQMNPCKNYLLQQCNPVSLVSSLVSMILPRSDCKVMRQQCCQQLARIPQQL

SEQ ID NO: 110 - protein, 381 aa, Triticum aestivum
ARELNPSEQELQQQQPRFQKGQQPFPQQSYPQQPYPSHQPFPTPQQYSPYQPQQPFPQPQQPTLIQPQQPFPQQPQQPQQPFPQPQQQLPLQPQQPFPQPQQPIPQQPQQSFPQQPQRPEQQFPQQPQQIIPQQTQQPFPLQPQQPFPQQPQRPFAQQPEQIISQQPFPLEPQQPSYQPQQPFPQQPGQIIPQQPQQPSPLQPQQPFSQQPQRPQQPFPQQPQQIIPQQPQQPFPLQPQQPVPQQPQRPFGQQPEQIISQRPQQPFPLQPQQPFSQPQQPFPQQPGQIIPQQPQQPFPLQPQQPFPQQPEQIIPQQPQQPFPLQPQQPFPQQPEQIIPQQPQQPFPLQPQQPSPQQPPHQQLPFPQPQQPFVSSGTSIGGQ

SEQ ID NO: 111 - protein, 354 aa, Triticum aestivum
MKPHHDGYKYTCSIIVTFHYPNFKHQDQKHQFQESIKHKSKMKTFIIFVLLSMPMSIVIAARHLNPSDQELQSPQQQFLEKTIISAATISTSTIFTTTTISHTPTIFPPSTTTTISPTPTTNPPTTTMTIPLATPTTTTTFSPAPTTISLATTTTISLAPTTNSPITTTTIPAATPETTTTIPPATRTNNYASTATTISLLTATTTPPATPTTILSATTTTISPAPTIISPATRTNNSLATPTTIPPATATTIPPATRTNNSPATATTIPPAPQQRFPHTRQKFPRNPNNHSLCSTHHFPAQQPFPQQPGQIIPQQPQQPLPLQPQQPFPWQPEQRSSQQPQQPFSLQPQQPFS

SEQ ID NO: 112 - protein, 345 aa, Triticum aestivum
ARQLNPSEQELQSPQQAVPKEQSYPQQPYPSHQPFPTPQQYSPYQPQQPFPQPQQPTPIQPQQPFPQQPQQPFPQPQQQLPLQPQQPFPQPQLPVSQQPQQPFPQQPQRPQQQFPQQPQQIIPQQTQQPFPLQPQQPFPQQPQRPFAQQPEQLISQQPFPLQPHQPFFQPQQPFPQQPGQIIPKQPQQPSTLQPQQPFSQQPQRPQQPFPQQPQQIIPQQPQQPFPLQPQQPVPQQPQRPFGQQPEQIISQRPQQPFPLQPQQPFSQPQQPLPQQPGQIIPQQPQQPFPLQPQQPFPQQSKQIIPQQPQQPFPLQPQQPSPQQPQLPFPQPQQPFVSSGTGIGGQ

SEQ ID NO: 113 - protein, 321 aa, Triticum aestivum
ARQLNPSEQELQSPQQAVPKEQSYPQQPQQPFPQPQQPTPIQPQQSFPQQPQQPFPQPQQQLPLQPQQPFPQPQQPIPQQPQQSYPQQPQRPQQQFLQQPQQIIPQQTQQPFPLQPQQPFPQQPQRPFAQQPEQIISQQPFPLQPQQLFSQPQQPFPQQPGQIINQQPQQPSPLQPQQPFSQQPQRPQQPFPQQPQQIIPPQPQQPFSLQPQQPVPQQPQRPFGQQPEQIISQRPQQPFPLQPKQPFSQPQQPFPQQPGQIIPQQPQQPFPLQPQQPFPQQPEQIISQQPQQPFPLQPQQPSHQQPQLPFPQPQQPFVVVE

SEQ ID NO: 114 - protein, 6 aa, Artificial Sequence
  Glutenin-like polypeptide
PGQGQQ

SEQ ID NO: 115 - protein, 9 aa, Artificial Sequence
  Glutenin-like polypeptide
GYYPTSPQQ

SEQ ID NO: 116 - protein, 838 aa, Triticum aestivum
MAKRLVLFVAVVVALVALTVAEGEASEQLQCERELQELQERELKACQQVMDQQLRDISPECHPVVVSPVAGQYEQQIVVPKGGSFYPGETTPPQQLQQRIFWGIPALLKRYYPSVTSPQQVSYYPGQASPQRPGQGQQPGQGQQSGQGQQGYYPTSPQQPGQWQQPEQGQPGYYPTSPQQPGQLQQPAQGQQPGQGQQGRQPGQGQPGYYPTSSQLQPGQLQQPAQGQQGQQPGQGQQGQQPGQGQQPGQGQQGQQPGQGQQPGQGQQGQQLGQGQQGYYPTSLQQSGQGQPGYYPTSLQQLGQGQSGYYPTSPQQPGQGQQPGQLQQPAQGQQPEQGQQGQQPGQGQQGQQPGQGQQPGQGQPGYYPTSPQQSGQGQPGYYPTSSQQPTQSQQPGQGQQGQQVGQGQQAQQPGQGQQPGQGQPGYYPTSPLQSGQGQPGYYLTSPQQSGQGQQPGQLQQSAQGQKGQQPGQGQQPGQGQQGQQPGQGQQGQQPGQGQPGYYPTSPQQSGQGQQPGQWQQPGQGQPGYYPTSPLQPGQGQPGYDPTSPQQPGQGQQPGQLQQPAQGQQGQQLAQGQQGQQPAQVQQGQQPAQGQQGQQLGQGQQGQQPGQGQQPAQGQQGQQPGQGQQGQQPGQGQQPGQGQPWYYPTSPQESGQGQQPGQWQQPGQWQQPGQGQPGYYLTSPLQLGQGQQGYYPTSLQQPGQGQQPGQWQQSGQGQHGYYPTSPQLSGQGQRPGQWLQPGQGQQGYYPTSPQQSGQGQQLGQWLQPGQGQQGYYPTSLQQTGQGQQSGQGQQGYYSSYHVSVEHQAASLKVAKAQQLAAQLPAMCRLEGGDALSASQ

SEQ ID NO: 117 - protein, 5 aa, Artificial Sequence
  Byssus-like polypeptide
GPGGG

(19) United States
(12) Patent Application Publication
(10) Pub. No.: US 2014/0206022 A1
(43) Pub. Date: Jul. 24, 2014

(54) THREE-DIMENSIONAL CELL CULTURE METHODS FOR TEST MATERIAL ASSESSMENT OF CELL DIFFERENTIATION

(71) Applicant: Allergan, Inc., Irvine, CA (US)

(72) Inventors: Gina M. Nuti, Santa Barbara, CA (US); Mark R. Verardo, Ventura, CA (US); Darin J. Messina, Santa Barbara, CA (US)

(73) Assignee: Allergan, Inc., Irvine, CA (US)

(21) Appl. No.: 14/108,182

(22) Filed: Dec. 16, 2013

Related U.S. Application Data
(60) Provisional application No. 61/738,125, filed on Dec. 17, 2012.

(51) Int. Cl. G01N 33/543

(52) U.S. Cl. CPC G01N 33/54306
USPC 435/7.92

(57) ABSTRACT

The present specification discloses three-dimensional in vitro cell-based methods to assess a test matrix polymer ability to support differentiation of a population of cells methods of screening a material for its ability to stimulate cell growth and/or differentiation.

This applications claims priority to and the benefit of U.S. Provisional Patent Application No. 61/738,125, filed Dec. 17, 2012, the entire disclosure of which is incorporated herein by this reference.

Autologous fat transfer (AFT), also known as fat grafting, is a process by which fat is harvested from one part of the body and injected into another part of the same person's body where additional volume is needed for cosmetic and/or aesthetic purposes. For those patients who do not have significant stores of fat tissue for procurement, alternative tissue engineering-based approaches include the harvest, expansion ex vivo, and re-injection of autologous or allogeneic stem cells for repopulating the region of interest, or the stimulation of endogenous stem cell migration and differentiation at the site of interest.

A common problem with fat grafting and related tissue engineered approaches is the poor survival of grafted cells or tissues leading to an unacceptable amount of volume loss or an unintended aesthetic outcome. In these procedures, loss of transplanted tissue volume over time as a result of its resorption by the body is a major problem. For example, transplantation of adipose tissue generally results in a loss of 20% to 90% of it volume one year after. This tissue loss is unpredictable and is a result of poor survival and/or regeneration from progenitor cells in the transplanted tissue due to necrosis and a lack of vascular formation. With respect to adipose tissue, tissue breakdown is associated with traumatic rupture of the cells, avascular necrosis, apoptosis of the adipocytes, inflammation secondary to apoptosis, fibrosis and contraction of the graft, and/or delipidation of the adipocytes with subsequent volume loss. Failed tissue grafts sometime produce stellate and irregular nodules with calcifications. As such, transplanted tissue methods are usually performed two or three times to obtain the desired effect, resulting in massive time and cost.

Given the significant quantity of multipotent stem cells in adipose tissue, one promising strategy to improve clinical outcomes of fat grafting is to augment the differentiation of engrafted or endogenous stem cells into native adipose tissue. In one possible approach, a biomaterial comprising components supportive of cell differentiation can be co-injected with fat and/or stem cells to stimulate adipocyte differentiation. Such a biomaterial would be a viable candidate for improving fat grafting and tissue-engineered approaches to new fat tissue formation.

Currently, the use of an in vivo animal model is the best approach for determining whether a candidate material can stimulate new adipose tissue formation. In this animal model system, stem cells and/or fat tissue must be injected with a test material into an animal for a period of 6 weeks or more, prior to removing the graft and evaluating the resultant tissue for evidence of adipogenesis. This approach can be costly, time consuming, and requires the use of animals.

Another possible approach for determining whether a candidate material can stimulate new adipose tissue formation is the use of standard cell culture models. In this in vitro cell culture model system, stem cells are cultured on two dimensional (2D) substrates that include a test material and a supportive extracellular matrix protein physically adsorbed to the surface of the cell culture plate. The cells are cultured in growth or cell maintenance media typically incubated at 37° C., and 5% CO₂, in a humidified incubator. After 3-7 days, the cells are evaluated for evidence of adipogenesis. Two-dimensional substrates, however, have limitations when it comes to predicting cell behavior or performance in a patient since tissues are three-dimensional in nature, and require nutrient flow, at a minimum, on both the apical and basal aspects of the cell.

To overcome the limitations of in vivo animal assays and/or in vitro 2D cell culture assays, the present specification discloses methods which use a three-dimensional (3D) culture model of stem cells with a candidate material. In these methods, cells are dispersed in a three-dimensional matrix comprising a test matrix polymer and/or test compound and incubated for a specified period of time in cell culture media supplemented with factors that promote differentiation of the seeded cells. The differentiation state of the cells is then determined and an assessment made as to whether the culture conditions support cell differentiation and new tissue formation. The disclosed methods allow for nutrient flow from at least two sides of the three-dimensional matrix. As such, the disclosed method mimics the three-dimensional environment to which the cells are exposed to in situ, and may therefore serve as a realistic, in vitro surrogate model. Moreover, the methods can be cost-effective, and be performed more quickly than an in vivo assay, facilitating the rapid screening of candidate matrix polymers, compounds, and/or other components for their ability to stimulate cell differentiation.

### SUMMARY

Aspects of the present specification disclose three-dimensional in vitro cell-based methods useful to assess the ability of a test matrix polymer to support differentiation of a population of cells. The disclosed methods comprise culturing a three-dimensional matrix comprising the test matrix polymer and the population of cells in a nutrient medium using an apparatus, wherein the three-dimensional matrix comprises a top surface and a bottom surface, and wherein the apparatus is configured to allow contact of the nutrient medium on the top surface and the bottom surface of the three-dimensional matrix; and assaying the population of cells for differentiation, wherein detection of differentiation is indicative that the test matrix polymer stimulates differentiation of a population of cells.

Other aspects of the present specification disclose methods of screening a material for its ability to stimulate cell growth and/or differentiation. The disclosed methods comprise incubating cells in a three-dimensional culture to determine growth of the cells; wherein the three-dimensional culture comprises the cells dispersed in a three-dimensional matrix comprising a material; and wherein the three-dimensional matrix is in contact with a nutrient medium such that a nutrient in the nutrient medium contacts from opposite sides the three-dimensional matrix of the biomaterial.

### BRIEF DESCRIPTION OF THE DRAWINGS

FIG. 1 shows an illustration of an apparatus used in the disclosed 3D in vitro cell-based method.

FIG. 2 shows an image taken from a fluorescence microscopy demonstrating adipocyte morphology. The arrow indicates a representative intracellular lipid droplet, a characteristic feature of mature adipocytes.

### DETAILED DESCRIPTION

Aspects of the present specification provide, in part, a three-dimensional matrix. A three-dimensional matrix, or matrix substrate refers to a three-dimensional structural framework comprising a matrix polymer. Typically, a three-dimensional matrix disclosed herein is designed to mimic the extracellular matrix of a multi-cellular organism. An exemplary example of a three-dimensional matrix is a hydrogel or gel.

Aspects of the present specification provide, in part, a three-dimensional matrix comprising a test matrix polymer. As used herein, the term “matrix polymer” refers to a polymer that can become part of a three-dimensional matrix. A matrix polymer may be a naturally-occurring polymer or a derivative thereof, a non-naturally-occurring or synthetic polymer or a derivative thereof, and pharmaceutically acceptable salts thereof. A matrix polymer may be a homopolymer, a copolymer, a random copolymer, a grafted co-polymer, a block co-polymer, or an interpenetrating network co-polymer. A test matrix polymer is simply a matrix polymer that is being assessed in the methods disclosed herein for its suitability to support growth and/or differentiation of cells.

Any matrix polymer suitable for use in a three-dimensional matrix may be employed as a test matrix polymer. Non-limiting examples of a test matrix polymer include a polysaccharide, a polypeptide, or a polyester. Exemplary polysaccharides include, without limitation, a cellulose, an agarose, a dextran, a xylogucan, a chitosan, a chitin, a starch, a glycosaminoglycan, or derivatives thereof. A cellulose derivative includes, e.g., methylcellulose (MC) and hydroxypropyl methylcellulose (HMC). Exemplary polypeptides include, without limitation, an elastic protein (including a silk protein, a resilin, a resilin-like polypeptides (RLPs), an elastin, an elastin-like polypeptides (ELPs), a silk protein-elastin-like polypeptides (SELPs), a gluten, an abductin, a byssus, a keratin, a gelatin, a lubricin, or a collagen. Exemplary polyesters include, without limitation, D-lactic acid, L-lactic acid, racemic lactic acid, glycolic acid, caprolactone. Non-limiting examples of a pharmaceutically acceptable salt of a matrix polymer includes sodium salts, potassium salts, magnesium salts, calcium salts, and combinations thereof.

Aspects of the present specification provide, in part, a three-dimensional matrix comprising a glycosaminoglycan. As used herein, the term “glycosaminoglycan” is synonymous with “GAG” and “mucopolysaccharide” and refers to long unbranched polysaccharides consisting of a repeating disaccharide units. The repeating unit consists of a hexose (six-carbon sugar) or a hexuronic acid, linked to a hexosamine (six-carbon sugar containing nitrogen) and pharmaceutically acceptable salts thereof. Members of the GAG family vary in the type of hexosamine, hexose or hexuronic acid unit they contain, such as, e.g., glucuronic acid, iduronic acid, galactose, galactosamine, glucosamine) and may also vary in the geometry of the glycosidic linkage. Non-limiting examples of glycosaminoglycans include chondroitin sulfate, dermatan sulfate, keratan sulfate, hyaluronan. Non-limiting examples of an acceptable salt of a glycosaminoglycans includes sodium salts, potassium salts, magnesium salts, calcium salts, and combinations thereof. Glycosaminoglycan and their resulting polymers useful in the methods disclosed herein are described in, e.g., Piron and Tholin, Polysaccharide Crosslinking, Hydrogel Preparation, Resulting Polysaccharides(s) and Hydrogel(s), uses Thereof, U.S. Patent Publication 2003/0148995; Lebreton, Cross-Linking of Low and High Molecular Weight Polysaccharides Preparation of Injectable Monophase Hydrogels; Lebreton, Viscoelastic Solutions Containing Sodium Hyaluronate and Hydroxypropyl Methyl Cellulose, Preparation and Uses, U.S. Patent Publication 2008/0089918; Lebreton, Hyaluronic Acid-Based Gels Including Lidocaine, U.S. Patent Publication 2010/0028438; and Polysaccharides and Hydrogels thus Obtained, U.S. Patent Publication 2006/0194758; and Di Napoli, Composition and Method for Intradermal Soft Tissue Augmentation, International Patent Publication WO 2004/073759, each of which is hereby incorporated by reference in its entirety.

Aspects of the present specification provide, in part, a three-dimensional matrix comprising a chondroitin sulfate polymer. As used herein, the term “chondroitin sulfate polymer” refers to an unbranched, sulfated polymer of variable length comprising disaccharides of two alternating monosaccharides of D-glucuronic acid (GlcA) and N-acetyl-D-galactosamine (GalNAc) and pharmaceutically acceptable salts thereof. A chondroitin sulfate polymer may also include D-glucuronic acid residues that are epimerized into L-iduronic acid (IdoA), in which case the resulting disaccharide is referred to as dermatan sulfate. A chondroitin sulfate polymer can have a chain of over 100 individual sugars, each of which can be sulfated in variable positions and quantities. Chondroitin sulfate polymers are an important structural component of cartilage and provide much of its resistance to compression. Non-limiting examples of pharmaceutically acceptable salts of chondroitin sulfate include sodium chondroitin sulfate, potassium chondroitin sulfate, magnesium chondroitin sulfate, calcium chondroitin sulfate, and combinations thereof.

Aspects of the present specification provide, in part, a three-dimensional matrix comprising a keratan sulfate polymer. As used herein, the term “keratan sulfate polymer” refers to a polymer of variable length comprising disaccharide units, which themselves include β-D-galactose and N-acetyl-D-galactosamine (GalNAc) and pharmaceutically acceptable salts thereof. Disaccharides within the repeating region of keratan sulfate may be fucosylated and N-Acetylneuraminic acid caps the end of the chains. Non-limiting examples of pharmaceutically acceptable salts of keratan sulfate include sodium keratan sulfate, potassium keratan sulfate, magnesium keratan sulfate, calcium keratan sulfate, and combinations thereof.

Aspects of the present specification provide, in part, a three-dimensional matrix comprising a hyaluronan polymer. As used herein, the term “hyaluronan polymer” is synonymous with “HA polymer”, “hyaluronic acid polymer”, and “hyaluronate polymer” refers to an anionic, non-sulfated glycosaminoglycan polymer comprising disaccharide units, which themselves include D-glucuronic acid and D-N-acetylglucosamine monomers, linked together via alternating β-1,4 and β-1,3 glycosidic bonds and pharmaceutically acceptable salts thereof. Hyaluronan polymers can be purified from animal and non-animal sources. Polymers of hyaluronan can range in size from about 5,000 Da to about 20,000,000 Da. Non-limiting examples of pharmaceutically acceptable salts of hyaluronan include sodium hyaluronan, potassium hyaluronan, magnesium hyaluronan, calcium hyaluronan, and combinations thereof.

In some embodiments, a hyaluronic acid may have a molecular weight of about 200,000 daltons to about 10,000,000 daltons, about 500,000 daltons to about 10,000,000 daltons, about 1,000,000 daltons to about 5,000,000 daltons, or about 1,000,000 daltons to about 3,000,000 daltons. When the crosslinking reaction occurs, the resulting crosslinked macromolecular product may have a hyaluronic acid component derived from the hyaluronic acid in the crosslinking reaction. Thus, the ranges recited above may also apply to the molecular weight of a hyaluronic acid component, e.g. about 200,000 daltons to about 10,000,000 daltons, about 500,000 daltons to about 10,000,000 daltons, about 1,000,000 daltons to about 5,000,000 daltons, or about 1,000,000 daltons to about 3,000,000 daltons. The term “molecular weight” may be applied to a matrix polymer that is solely hyaluronic acid or another matrix polymer, or it may be used to describe the mass of a matrix polymer that is linked to another matrix polymer, such as in a macromolecular matrix comprising a hyaluronic acid crosslinked to another matrix polymer, such as a collagen. For example, the term molecular weight may be applied to the hyaluronic acid and/or the collagen to indicate the size of the precursor matrix polymers of the matrix.

Aspects of the present specification provide, in part, a three-dimensional matrix comprising an elastic protein. As used herein, the term “elastic protein” is synonymous with “bioelastomer” and refers to a polypeptide possessing rubber-like elasticity. An elastic protein can undergo high deformation without rupture, storing the energy involved in deformation and then returning to its original state when the stress is removed. The latter phase is passive and returns all, or nearly all, of the energy used in deformation. As such, an elastic protein has high resilience in that the polypeptide can be deformed reversibly without little loss of energy. Additionally, an elastic protein can be deformed to large strains with little force, and/or has low stiffness in that the polypeptide can be stretched. In general, properties useful to characterize elastic protein include stiffness, as evaluated by the modulus of elasticity (Einit, Nm-2); strength, as evaluated by the stress at fracture (σmax, Nm-2); toughness, as evaluated by the energy to break work of fracture (Jm-3, Jm⁻²); extensibility, as evaluated by the strain at fracture (εmax, no units); spring efficiency, as evaluated by resilience (%); durability, as evaluated by lifetime fatigue (s to failure or cycles of failure); and spring capacity, as evaluated by energy storage capacity (Wout, Jkg-1). For example, elastic proteins like elastin and resilin have a combination of high resilience, large strains and low stiffness is characteristic of rubber-like proteins that function in the storage of elastic-strain energy. Other elastic proteins, like collagens, provide exceptional energy storage capacity but are not very stretchy. Mussel byssus threads and spider dragline silks are also elastic proteins because they are remarkably stretchy, in spite of their considerable strength, low resilience, and stiffness.

Non-limiting examples of elastic proteins include silk proteins, resilins, resilin-like polypeptides (RLPs), elastins (including tropoelastin, fibrillin and fibullin), elastin-like polypeptides (ELPs), glutens (including gliadins and glutenins), abductins, byssuss, and collagens. In general, elastic proteins have at least one domain containing elastic repeat motifs and another non-elastic domain where crosslinks can be formed. See, e.g., Tatham and Shewry, Comparative Structures and Properties of Elastic Proteins, Phil. Trans. R. Soc. Lond. B 357: 229-234 (2002), which is hereby incorporated by reference in its entirety. However, both resilin and abductin are exceptions since crosslinking can occur within the elastic repeat motif.

Silk protein refers to a filamentous product secreted by an organism such as a spider or silkworm. Fibroin is the primary structural component of silk. It is composed of monomeric units comprising an about 350 kDa heavy chain (see, e.g., SEQ ID NO: 1, SEQ ID NO: 65) and an about 25 kDa light chain (see, e.g., SEQ ID NO: 2, SEQ ID NO: 66), and interspersed within the fibroin monomers is another about 25 kDa protein (see, e.g., SEQ ID NO: 67) derived from the P25 gene.

Resilin is found in specialized regions of the cuticle of most insects, providing low stiffness, high strain and efficient energy storage; it is best known for its roles in insect flight and the remarkable jumping ability of fleas and spittle bugs. It has no regular structure but its randomly coiled chains are crosslinked by di- and tri-tyrosine links at the right spacing to confer elasticity. Resilin must last for the lifetime of adult insects and must therefore operate for hundreds of millions of extension and contraction; its elastic efficiency ensures performance over the insect's lifetime. Resilin exhibits unusual elastomeric behavior only when swollen in polar solvents such as water. The soluble precursor of resilin is proresilin. Proresilin is about 600 amino acids in length and has an amino-terminal domain comprising one type of elastic repeat motifs, a central non-repetitive domain, and an amino-terminal domain comprising another type of elastic repeat motifs. In insects, proresilin is secreted in the subcuticular space where it undergoes rapid crosslinking at tyrosine residues, through di- and trityrosine crosslink formations. Crosslinking appears to involve enzymatic reactions involving peroxidases. Exemplary resilin amino acid sequences include SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23.

Resilin fragments comprising elastic repeat motifs as well as fragments comprising the amino acid segment encoded by first exon produce resilin proteins useful as compositions and in the methods disclosed herein. Resilin, and resilin fragments useful to the compositions and methods disclosed herein, can be produced recombinantly by expressing a genetic construct encoding this protein in a standard expression system like a bacterial, yeast, insect or mammalian expression system and purifying the resulting resilin using routine procedures. Such expression constructs encoding resilin and functional resilin fragments and purification methods are described in, e.g., Elvin, et al., Synthesis and Properties of Crosslinked Recombinant Proresilin, Nature 437: 999-1002 (2005); Lyons, et al, Design and Facile Production of Recombinant Resilin-Like Polypeptides: Gene construction and a Rapid Protein Purification Method, Protein Eng. Des. Sel. 20: 25-32 (2007); Nairn, et al., A Synthetic Resilin is Largely Unstructured, Biophys. J. 95: 3358-3365 (2008), each of which is incorporated by reference in its entirety. Resilin can be crosslinked using standard procedures, like rapid photochemical, to produce a resilin hydrogel. Such a resilin hydrogel can be processed to contain additional components such as, e.g., amphiphilic and synthetic peptides disclosed herein, protease cleavage sites to facilitate biodegradation, and used in a manner and in the methods as disclosed herein for a silk fibroin hydrogel.

Resilin-like polypeptides (RLPs) are derived from an elastic repeat motif found within resilin and can be 5 to 1,500 amino acids in length. The most common elastic repeat motifs include YGAP (SEQ ID NO. 24), AQTPSSQYGAP (SEQ ID NO. 25), GGRPSDSYGAPGGGN (SEQ ID NO. 26), GYSGGRPGGQDLG (SEQ ID NO. 27), PGGGN (SEQ ID NO. 28), PGGGNGGRP (SEQ ID NO. 29), SDTYGAPGGGNGGRP (SEQ ID NO. 30), and PGGGNGGRPSDTYGAPGGGNGGRP (SEQ ID NO. 31). In one embodiment, the RLP has the general formula of (SEQ ID NO. 24)m, (SEQ ID NO. 25)m, (SEQ ID NO. 26)m, (SEQ ID NO. 27)m, (SEQ ID NO. 28)m, (SEQ ID NO. 29)m, (SEQ ID NO. 30)m, and (SEQ ID NO. 31)m, or any combination thereof, where m is the number of repeats comprising the RLP. In an aspect of this embodiment, m is 0-200. RLPs comprising these elastic repeat motifs exhibit properties similar to resilin. RLPs can be designed at the molecular level and genetically synthesized to add unique properties that can be introduced by incorporating other biologically active peptide sequences. As such, RLP hydrogels can be formed by crosslinking using a variety of methods including, without limitation, irradiation, photoinitiation, amine-reactive chemical crosslinking and enzymatic crosslinking. Such an RLP hydrogel can be processed to contain additional components such as, e.g., amphiphilic and synthetic peptides disclosed herein, protease cleavage sites to facilitate biodegradation, and used in a manner and in the methods as disclosed herein for a silk fibroin hydrogel. Exemplary RLP amino acid sequences include SEQ ID NO. 32, SEQ ID NO. 33, and SEQ ID NO. 34. Other RLPs are described in, e.g., Elvin, Bioelastomers, U.S. Patent Publication 2007/0099231 and Elvin, Synthetic Bioelastomers, U.S. Patent Publication 2007/0275408, each of which is hereby incorporated by reference in its entirety.

One of the most abundant extracellular matrix proteins, elastin is an insoluble crosslinked polymer that forms massive complex arrays. Elastin is composed of monomeric subunits of a soluble precursor called tropoelastin that has a molecular weight of about 66-70 kDa. Tropoelastin is about 760 amino acids in length and composed of alternating hydrophobic domains rich in glycine, valine and praline residues; and hydrophilic domains rich in lysine and arginine residues. Elastin is formed and stabilized by crosslinking tropoelastin monomers at lysine residues, in a reaction catalyzed by lysyl oxidase or transglutaminase. Exemplary tropoelastin amino acid sequences include SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, or SEQ ID NO: 55. Like proresilin, tropoelastin can be recombinantly made by expressing a genetic construct encoding this protein in a standard expression system and purifying the resulting tropoelastin using routine procedures. Such expression constructs encoding tropoelastin and functional tropoelastin fragments and purification methods are described in, e.g., Urry, et al., Elastic Protein-Based Polymers in Soft Tissue Augmentation and Generation, J. Biomater. Sci. Polym. Ed. 9: 1015-1048 (1998), which is hereby incorporated by reference in its entirety. These monomeric subunits can then be enzymatically crosslinked using lysyl oxidase or transglutaminase to form an elastin hydrogel. See, e.g., Betre, et al., Characterization of a Genetically-Engineered Elastin-Like Polypeptide for Cartilaginous Tissue Repair, Biomolecules 3: 910-916 (2003); Ong, Epitope-Tagging for Tracking Elastin-Like Polypeptides, Biomaterials 27: 1930-1935 (2006); Strokowski and Woodhouse, Development and Characterization of Novel Cross-Linked Bioelatomeric Materials, J. Biomater. Sci. Polym. Ed. 19: 785-799 (2008), each of which is incorporated by reference in its entirety. Such a elastin hydrogel can be processed to contain additional components such as, e.g., amphiphilic and synthetic peptides disclosed herein, protease cleavage sites to facilitate biodegradation, and used in a manner and in the methods as disclosed herein for a silk fibroin hydrogel.

Elastin-like polypeptides (ELPs) can be 5 to 1,500 amino acids in length and are generally made from an elastic repeat motif found within a hydrophobic domain of tropoelastin. See, e.g., Banta, et al., Protein Engineering in the Development of Functional Hydrogels, Annu. Rev. Biomed. Eng. 12: 167-186 (2010), which is hereby incorporated by reference in its entirety. The most common elastic motif has the amino acid sequence VPGXG (SEQ ID NO: 56), where X can be any amino acid other than proline. However, other elastic repeat motifs include KGGVG (SEQ ID NO: 57), LGGVG (SEQ ID NO: 58), LGAGGAG (SEQ ID NO: 59), and LGAGGAGVL (SEQ ID NO: 60), where m is the number of repeats comprising the ELP. Any combination of these elastin elastic repeat motifs can be used to design an ELP. In one embodiment, the ELP has the general formula of (SEQ ID NO: 56)m, (SEQ ID NO: 57)m, (SEQ ID NO: 58)m, (SEQ ID NO: 59)m, and (SEQ ID NO: 60)m, or any combination thereof, where m is the number of repeats comprising the ELP. In an aspect of this embodiment, m is 0-200. In an aspect of this embodiment, an ELP has the formula (m) (SEQ ID NO: 61) (SEQ ID NO: 56)mWP, where X is Valine, Alanine, or Glycine in a ratio of 5:2:3 and m is 1-200. In another aspect of this embodiment, an ELP has the formula (m) (SEQ ID NO: 61) (SEQ ID NO: 56)mWP, where X is Valine, Alanine, or Glycine in a ratio of 1:8:7 and m is 1-200. In yet another aspect of this embodiment, an ELP has the formula (m) (SEQ ID NO: 61) (SEQ ID NO: 56)mWP, where X is Valine, Isoleucine, or Glutamine in a ratio of 1:3:1 and m is 1-200. ELPs comprising these repeating motifs exhibit elastin-like properties. Exemplary ELP amino acid sequences include SEQ ID NO: 62, SEQ ID NO: 63, and SEQ ID NO: 64. Other ELPs are described in, e.g., Masters, Protein Matrix Materials, Devices and Methods of Making and Using Thereof, U.S. Pat. No. 7,662,409; Chaikof, et al., Native Protein Mimetic Fibers, Fiber Networks and Fabrics for Medical Use, U.S. Patent Publication 2004/0110439, each of which is hereby incorporated by reference in its entirety.

ELPs are highly soluble in an aqueous solution below their transition temperature (Tt), but aggregate rapidly above their Tt in a process called inverse phase transition. ELPs are good candidates for chemical crosslinking because a chemically active amino acid, like lysine or glutamine, can be easily to incorporate into the X site of the repeating motif. In addition, because ELPs can be designed at the molecular level and genetically synthesized, unique properties can be introduced by incorporating other biologically active peptide sequences. As such, ELP hydrogels can be formed by irradiation, photoinitiation, amine-reactive chemical crosslinking and enzymatic crosslinking. Like tropoelastin, ELPs can be recombinantly made by expressing a genetic construct encoding this protein in a standard expression system and purifying the resulting tropoelastin using routine procedures. Such ELPs, expression constructs encoding ELPs purification methods, and crosslinking procedures are described in, e.g., Urry, et al. Elastic protein-based polymers in soft tissue augmentation and generation, J. Biomater. Sci. Polym. Ed. 9(10): 1015-1048 (1998); Betre, et al., Characterization of a genetically engineered elastin-like polypeptide for cartilaginous tissue repair, Biomacromolecules 3(5): 910-916 (2002); Haider, et al., Molecular engineering of silk-elastinlike polymers for matrix-mediated gene delivery: biosynthesis and characterization, Mol. Pharm. 2(2): 139-150 (2005); McHale, et al., Synthesis and in vitro evaluation of enzymatically cross-linked elastin-like polypeptide gels for cartilaginous tissue repair, Tissue Eng. 11(11-12): 1768-1779 (2005); Srokowski and Woodhouse, Development and characterisation of novel cross-linked bio-elastomeric materials, J. Biomater. Sci. Polym. Ed. 19(6): 785-799 (2008); and MacEwan and Chilkoti, Elastin-Like Polypeptides: Biomedical Applications of Tunable Biopolymers, Peptide Sci. 94(1): 60-77 (2010), each of which is hereby incorporated by reference in its entirety. Such an ELP hydrogel can be processed to contain additional components such as, e.g., amphiphilic and synthetic peptides disclosed herein, protease cleavage sites to facilitate biodegradation, and used in a manner and in the methods as disclosed herein for a silk fibroin hydrogel.

Silk-elastin-like polypeptides (SELPs) comprise tandem repeats of silk-like elastic repeat motifs and elastin elastic repeat motifs. See, e.g., Haider, et al., Molecular Engineering of Silk-Elastinlike Polymers for Matrix-Mediated Gene Delivery: Biosynthesis and Characterization, Mol. Pharmaceutics. 2(2): 139-150 (2005), which is hereby incorporated by reference in its entirety. The most common elastic motif from silk proteins has the amino acid sequence (GAGAGS)m, (SEQ ID NO: 68), where m is the number of repeats comprising the SELP, whereas elastic motif from elastins are as disclosed herein. Other elastic motifs from silk proteins useful in designing a SELP include, without limitation, GAAGY (SEQ ID NO: 69), AGAGAGPEG (SEQ ID NO: 70), AGAGAGEG (SEQ ID NO: 71), GAGAGSGAAGGAGAGSGAGAGSGAGAGSGAGAGS GAGAGSGAGAGSGAGAGSGAGAGSY (SEQ ID NO: 72), and YGGLGSQGAGRGG (SEQ ID NO: 73). By combining the silk and elastin elastic motifs in various ratios and sequences, it is possible to produce a variety of SELPs with diverse material properties. The formation of hydrogen binds between the silk elastic motifs appears to be the primary driving force behind gelation. The inclusion of elastin elastic motifs increases flexibility and aqueous solubility of the SELP. Exemplary SELP amino acid sequences include SEQ ID NO: 74, SEQ ID NO: 75, SEQ ID NO: 76, and SEQ ID NO: 77. Other SELPs as described in, e.g., Masters, Protein Matrix Materials, Devices and Methods of Making and Using Thereof, U.S. Pat. No. 7,662,409; Cappello, Synthetic Protein as Implantables, U.S. Pat. No. 5,606,019, Kumar, et al., Controlled Release of Active Agents Utilizing Repeat Sequence Protein Polymers, U.S. Patent Publication 2004/0228913, Kumar, et al., Use of Repeat Sequence Protein Polymers in Personal Care Compositions, U.S. Patent Publication 2005/0142094, Collier, et al., Repeat Sequence Protein Polymer Active Ingredient Conjugates, Methods and Uses, U.S. Patent Publication 2006/0153791, each of which is hereby incorporated by reference in its entirety.

SELPs are good candidates for chemical crosslinking because a chemically active amino acid, like lysine or glutamine, can be easily to incorporate into the X site of the repeating elastin elastic motif. In addition, because SELPs can be designed at the molecular level and genetically synthesized, unique properties can be introduced by incorporating other biologically active peptide sequences. As such, ELP hydrogels can be formed by irradiation, photoinitiation, amine-reactive chemical crosslinking and enzymatic crosslinking. Like tropoelastin, SELPs can be recombinantly made by expressing a genetic construct encoding this protein in a standard expression system and purifying the resulting tropoelastin using routine procedures. SELPs can be crosslinked using standard procedures, like rapid photochemical, to produce a SELP hydrogel. Such a SELP hydrogel can be processed to contain additional components such as, e.g., amphiphilic and synthetic peptides disclosed herein, protease cleavage sites to facilitate biodegradation, and used in a manner and in the methods as disclosed herein for a silk fibroin hydrogel.

Abductin is a rubber-like protein from the internal triangular hinge ligament of bivalve mollusks, acting as an elastic pivot that antagonizes the action of the adductor muscle. Abductin is an about 136 residue polypeptide comprising two domains. An alanine-rich amino-terminal domain of 20 residues in length contains two tyrosine residues believed to be involved in crosslinking. The second domain comprises 11 glycine-methionine-rich decapeptide repeats. This 10 amino acid elastic repeat motif has the acid sequence GGFGGMGGGX (SEQ ID NO: 78), where X is any amino acid. Exemplary Abductin amino acid sequences include SEQ ID NO: 79, SEQ ID NO: 80, SEQ ID NO: 81, and SEQ ID NO: 82.

Gluten comprises two proteins, gliadin and glutenin that exist, conjoined with starch, in the endosperms of some grass-related grains, notably wheat, rye, and barley. Gliadins are glycoprotein present in wheat and several other cereals within the grass genus Triticum. Gliadins are prolamins that are slightly soluble in ethanol, and are separated on the basis of electrophoretic mobility and isoelectric focusing, with α-β-gliadins, γ-gliadins, and ω-gliadin. Exemplary gliadin amino acid sequences include SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, and SEQ ID NO: 113.

Glutenin consists of 20% High-Molecular-Weight (HMW) subunits, which are relatively low in sulfur and 80% are Low-Molecular-Weight (LMW) subunits and are high in sulfur. The HMW subunit is about 825 amino acids in length and comprises a large central repetitive domain comprising hexapeptide PGQGQQ (SEQ ID NO: 114), nonapeptide GYYPTSPQQ (SEQ ID NO: 115), and tripeptide GQQ elastic repeat motifs. Because it is insoluble in water, gluten can be obtained by simply washing slurry of flour in water by stirring vigorously to dissolve the associated starch. The resulting gummy mass, which is about 70% to about 80% gluten, may then be centrifuged to collect the gluten. If a saline solution is used instead of water a purer gluten fraction is obtained. Gluten is also commercially available. Exemplary gliadin amino acid sequences include SEQ ID NO: 116.

Byssus is a major protein component present in the byssal threads used to attach mussels to hard surfaces in water. One form of byssus, Col-P comprises a central collagen-like domain of about 430 amino acids flanked by an amino-terminal elastic domain of about 100 amino acids and by a carboxyl-terminal elastic domain of about 160 amino acids. See, e.g., Tatham and Shewry, Comparative Structures and Properties of Elastic Proteins, Phil. Trans. R. Soc. Lond. B 257: 229-234 (2002), which is hereby incorporated by reference in its entirety. The elastic domains comprise a pentapeptide repeat motif and histidine-rich domains. This 5 amino acid elastic repeat motif has the acid sequence GPGGG (SEQ ID NO: 117).

Collagen is a protein that forms fibrils and sheets that bear tensile loads. Collagen also has specific integrin-binding sites for cell adhesion and is known to promote cell attachment, migration, and proliferation. The collagen superfamily contains at least 29 different types of collagen, designated COL1A1-COL29A1. Some collagens have several isoforms, such as, e.g., COL1A1, COL1A2, COL4A1, COL4A2, COL4A3, COL4A4, COL4A5, COL4A6, COL5A1, COL5A2, COL5A3, COL6A1, COL6A2, COL6A3, COL8A1, COL8A2, COL9A1, COL9A2, COL9A3, COL11A1, and COL11A2. Collagens are found in all connective tissue and are a major component of the extracellular matrix. Collagens can be purified from animal sources, plant sources, or produced recombinantly. Although 29 types of collagen have been identified, over 90% of the collagen in the body is of type I, II, III, and IV. Collagen I is found in skin, tendon, vascular, ligature, organs, and is the main component of bone; collagen II is the main component of cartilage; collagen III is the main component of reticular fibers; collagen IV forms bases of cell basement membrane; and collagen V is present on cells surfaces, hair and placenta. Gelatin is a protein produced by partial hydrolysis of collagen extracted from the boiled bones, connective tissues, organs and intestines of animals such as cattle, pigs, and horses. Collagens are also commercially available. The elastic domain comprises a tripeptide repeat motif of either GXP or GXHyp, where X is any amino acid and Hyp is hydroxyproline. Collagen-based elastic proteins are described in, e.g., Masters, Protein Matrix Materials, Devices and Methods of Making and Using Thereof, U.S. Pat. No. 7,662,409, which is hereby incorporated by reference in its entirety. Collagen may be positively charged because of its high content of basic amino acid residues such as arginine, lysine, and hydroxylysine. Unless clearly indicated otherwise, reference to collagen herein may include uncharged collagen, as well as any cationic forms, anionic forms, or salts of collagen.

Other elastic proteins useful in the compositions and methods disclosed herein are described in, e.g., Masters, Protein Matrix Materials, Devices and Methods of Making and Using Thereof, U.S. Pat. No. 7,662,409; and Kaplan, et al., Fibrous Protein Fusions and Use Thereof in the Formation of Advanced Organic/Inorganic Composite Materials, U.S. Patent Publication 2008/0293919, each of which is hereby incorporated by reference in its entirety.

Aspects of the present specification provide, in part, a three-dimensional matrix comprising a polyester. Included among polyesters of interest are homo- or copolymers of aliphatic polyesters like D-lactide, L-lactide, or racemic lactide polymers, collectively referred to as PLA polymers; D-glycolide, L-glycolide, or racemic glycolide polymers, collectively referred to as PGA polymers; poly(D,L or L-lactide-co-glycolide) copolymers, collectively referred to as PLGA copolymers; poly(ε-caprolactone) (PCL) polymers, and combinations thereof. For some copolymers of glycolide and lactide, biodegradation may be affected by the ratio of glycolide to lactide.

A three-dimensional matrix disclosed herein can include a single matrix polymer or a plurality of matrix polymers. In the case where there are two or more matrix polymers, one, more than one, or all of the polymers may be a test matrix polymer. In aspects of this embodiment, a three-dimensional matrix may comprise, e.g., two matrix polymers, three matrix polymers, four matrix polymers, five matrix polymers, or six matrix polymers. In other aspects of this embodiment, a three-dimensional matrix may comprise, e.g., at least two matrix polymers, at least three matrix polymers, at least four matrix polymers, at least five matrix polymers, or at least six matrix polymers. In yet other aspects of this embodiment, a three-dimensional matrix may comprise from between, e.g., one and two matrix polymers, one and three matrix polymers, one and four matrix polymers, one and five matrix polymers, one and six matrix polymers, two and three matrix polymers, two and four matrix polymers, two and five matrix polymers, two and six matrix polymers, three and four matrix polymers, three and five matrix polymers, or three and six matrix polymers. Whether present in the three-dimensional matrix as a single polymer or a plurality of polymers, the matrix polymers may be synthetically linked to one another.

For three-dimensional matrix comprising a hyaluronic acid cross-linked to a collagen, any suitable weight ratio of the hyaluronic acid component to the collagen component may be used. For example, a crosslinked macromolecular matrix may have a weight ratio of hyaluronic acid:collagen of about 1:2 to about 10:1, about 1:1 to about 7:1, about 2:1 to about 3:1, about 1:1 to about 3:1, about 1:1 to about 2:1, about 1:1, about 2:1, about 3:1, about 7:2, about 4:1, about 5:1, 16:3, about 6:1, about 7:1, or any weight ratio in a range bounded by, and/or between, any of these values. In some embodiments, the weight ratio of hyaluronic acid to collagen in a crosslinked matrix may be about 12 mg/mL of hyaluronic acid to about 6 mg/mL collagen, about 12 mg/mL of hyaluronic acid to about 12 mg/mL collagen, or about 16 mg/mL of hyaluronic acid to about 8 mg/mL collagen. In some embodiments, the collagen may be collagen type 1.

Aspects of the present specification provide, in part, a three-dimensional matrix comprising a test compound. A test compound is simply a compound that is being assessed in the methods disclosed herein for its suitability to support growth and/or differentiation of cells. A test compound includes, without limitation, a nutrient, a serum, an antibiotic, a supplement, a growth factor, or a differentiation factor. Non-limiting examples of a test compound include a salt, such as, e.g., sodium chloride, sodium phosphate, calcium chloride; a saccharide, such as, e.g., a monosaccharide, a disaccharide, a trisaccharide, an oligosaccharide and a polysaccharides; an animal protein hydrolysate, such as, e.g., an amino acid, a dipeptide, a tripeptide, or an oligopeptide, or a polypeptide; a selenium; a thiol, such as, e.g., 2-mercaptoethanol, 1-thioglycerol; a lipids, such as, e.g., an animal derived lipid, a chemically defined lipid, a human lipid; a vitamin, such as, e.g., vitamin A, vitamin D, vitamin D, vitamin K, vitamin C, vitamin B1, vitamin B2, vitamin B3, vitamin B5, vitamin B6, vitamin B12, biotin, choline, folate; a mineral, such as, e.g., calcium, phosphorus, iron, iodine, zinc, copper, manganese, chromium, selenium, molybdenum, potassium, sodium, boron, germanium, silica, sulfur, vanadium; a serum, such as, e.g., a fetal bovine serum; an albumin, such as, e.g., a bovine serum albumin, a human serum albumin, a recombinant human serum albumin; an insulin, such as, e.g., a recombinant human insulin, a human insulin; a transferrin, such as, e.g., a human transferrin, a recombinant human transferring; a polyvinyl alcohol.

Aspects of the present specification provide, in part, a three-dimensional matrix comprising a test material. A test material includes, without limitation a test matrix disclosed herein and a test compound disclosed herein.

Aspects of the present specification provide, in part, a population of cells. Any type of cell population or cells may be used in the methods disclosed herein. Three basic categories of cells make up the mammalian body; germ cells, somatic cells, and stem cells. Germ cells are cell that gives rise to the gametes of an organism that reproduces sexually. Somatic cells are differentiated cells comprising the body of a multicellular organism. Stem cells are undifferentiated cells present in the body of a multicellular organism. Stem cells can divide with the potential to differentiate into a variety of other cell types that perform one or more specific functions and has the ability to self-renew. In mammals, there are two broad types of stem cells: embryonic stem cells, which are isolated from the inner cell mass of blastocysts, and adult stem cells, which are found in various tissues.

The potency of the stem cell refers to the extent or degree a stem cell can differentiate into different cell types. Totipotent (or omnipotent) stem cells can differentiate into embryonic and extraembryonic cell types and such cells can construct a complete, viable organism. Pluripotent stem cells can differentiate into nearly all cells derived from any of the three germ layers. Multipotent stem cells can differentiate into a number of cell types, but only those of a closely related family of cells. Oligopotent stem cells can differentiate into only a few cell types belonging to a closely related family of cells. Unipotent cells can produce only one cell type, their own, but have the property of self-renewal, which distinguishes them from non-stem cells. Multipotent, oligopotent and unipotent are referred to as lineage-restricted stem cells. Most adult stem cells are lineage-restricted and are generally referred to by their tissue origin. Exemplary examples of multipotent stem cells include, without limitation, adipose-derived stem cells (ASCs; adipose-derived stromal cells), endothelial-derived stem cells (ESCs), hemopoietic stem cells (HSGs), and mesenchyma stem cells (MSCs). Exemplary examples of oligopotent stem cells include, without limitation, endothelial progenitor cells, keratinocytes, monoblasts, myoblasts, and pericytes. Exemplary examples of unipotent stem cells include, without limitation, adipoblast (lipoblast or preadipocytes), de-differentiated adipocytes, angioblasts, endothelial precursor cells, fibroblasts, lymphoblasts, and macrophages.

Selection of cells used in the methods disclosed herein depend, in part, upon the type differentiation cells and/or tissue sought. For example, adipose-derived stem cells or adipoblasts may be seeded in a three-dimensional matrix when adipocytes are desired.

Aspects of the present specification provide, in part, a nutrient medium. A nutrient medium is any medium that can support cell viability including, e.g., cell metabolism, cell growth, cell differentiation, or any combination thereof. A nutrient medium is typically a liquid or a gel. Non-limiting examples of a nutrient medium include a cell culture medium like Delbecco's Modified Eagle Medium (DMEM), Eagle Medium, Ham's Medium, Iscove's Modified Delbecco's Medium (IMDM), RPMI 1640, and Minimum Essential Medium (MEM).

A nutrient media may optionally comprise one or more reagents like a nutrient, a serum, an antibiotic, a supplement, a growth factor, or a differentiation factor. Conversely, a nutrient media may optionally lack one or more reagents like a nutrient, a serum, an antibiotic, a supplement, a growth factor, or a differentiation factor.

Non-limiting examples of a reagent include a salt, such as, e.g., sodium chloride, sodium phosphate, calcium chloride; a saccharide, such as, e.g., a monosaccharide, a disaccharide, a trisaccharide, an oligosaccharide and a polysaccharides; an animal protein hydrolysate, such as, e.g., an amino acid, a dipeptide, a tripeptide, or an oligopeptide, or a polypeptide; a serum, such as, e.g., a fetal bovine serum; an albumin, such as, e.g., a bovine serum albumin, a human serum albumin, a recombinant human serum albumin; an insulin, such as, e.g., a recombinant human insulin, a human insulin; a transferrin, such as, e.g., a human transferrin, a recombinant human transferring; a polyvinyl alcohol; a selenium; a thiol, such as, e.g., 2-mercaptoethanol, 1-thioglycerol; a lipids, such as, e.g., an animal derived lipid, a chemically defined lipid, a human lipid; a vitamin, such as, e.g., vitamin A, vitamin D, vitamin D, vitamin K, vitamin C, vitamin B1, vitamin B2, vitamin B3, vitamin B5, vitamin B6, vitamin B12, biotin, choline, folate; a mineral, such as, e.g., calcium, phosphorus, iron, iodine, zinc, copper, manganese, chromium, selenium, molybdenum, potassium, sodium, boron, germanium, silica, sulfur, vanadium.

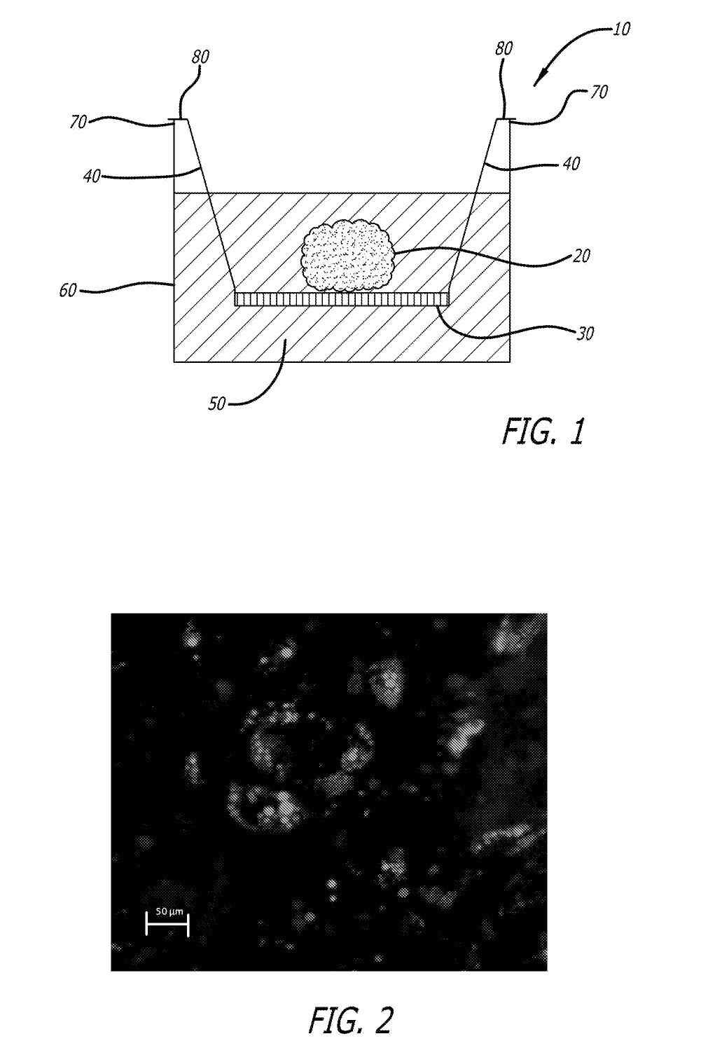

Aspects of the present specification provide, in part, culturing a three-dimensional matrix disclosed herein using an apparatus. One way to mimic the three-dimensional environment to which cells are exposed to in situ is to employ an apparatus configured to allow contact of the nutrient medium on the top and bottom surface of the three-dimensional matrix. In some embodiments, the desired contact may be achieved by suspending a three-dimensional matrix disclosed herein in a nutrient medium on a permeable support. While there may be many ways that this can be accomplished, FIG. 1 depicts an example of an apparatus that may be used to suspend a three-dimensional matrix in a nutrient medium on a permeable support in order to allow contact of the nutrient medium on the top and bottom surface of the three-dimensional matrix. In FIG. 1, device 10 may comprise a three-dimensional matrix 20 disposed on permeable support 30. Permeable support 30 may be coupled to suspension element 40, so that suspension element 40 keeps permeable support 30 suspended in liquid 50. Liquid 50, suspension element 40, permeable support 30, and three-dimensional matrix 20 may be contained within vessel 60. Vessel 60 may include rim 70 at the opening of vessel 60. Lip 80 may be attached to suspension element 40 so as to allow the entire assembly of suspension element 40, permeable support 30, and three-dimensional matrix 20, to be supported by allowing lip 80 to rest upon rim 70.

A permeable support, such as permeable support 30, may be composed of any of a variety of materials. Some permeable supports may comprise a polymeric material such as a polyester, a polycarbonate, a polytetrafluororethylene, or a similar material. A biomaterial, such as collagen or a similar material, may also be used. In some embodiments, a permeable support may comprise a polyester material, a polycarbonate material, a polytetrafluoroethylene material, or a collagen material.

A permeable support may include pores to allow nutrient medium to penetrate through the permeable support and make contact with the surface of the three-dimensional matrix disclosed herein. The pores may have any suitable size. In aspects of this embodiment, a pore may have a diameter of at least 0.1 μm, at least 0.25 μm, at least 0.5 μm, at least 0.75 μm, at least 1 μm, at least 2.5 μm, at least 5 μm, at least 7.5 μm, at least 10 μm, at least 15 μm, at least 20 μm, at least 25 μm, or at least 30 μm. In aspects of this embodiment, a pore may have a diameter of about 0.1 μm to about 30 μm, 0.2 μm to about 10 μm, about 0.4 μm to about 8 μm, about 0.4 μm, about 1 μm, about 3 μm, about 8 μm, or any diameter in a range bounded by, or between, any of these values.

A permeable support may have any suitable thickness. In aspects of this embodiment, a permeable support may have a thickness of at least 1 μm, at least 2.5 μm, at least 5 μm, at least 7.5 μm, at least 10 μm, at least 25 μm, at least 50 μm, at least 75 μm, or at least 100 μm. In other aspects of this embodiment, a permeable support may have a thickness of about 1 μm to about 100 μm, about 5 μm to about 70 μm, about 10 μm to about 50 μm, about 10 μm, or about 50 μm.

A three-dimensional matrix disclosed herein may be placed in contact with a nutrient medium in any manner that allows nutrients to flow from opposite sides of the three-dimensional matrix. In one embodiment, a three-dimensional matrix may be fully immersed in the nutrient medium. In another embodiment, a three-dimensional matrix may be partially immersed in the nutrient medium. In aspects of this embodiment, a three-dimensional matrix may be partially immersed in the nutrient medium so that, e.g., at least 50%, at least 55%, at least 60%, at least 65%, at least 70%, at least 75%, at least 80%, at least 85%, at least 90%, at least 95%, at least 97%, or at least 99% of the three-dimensional matrix may be in contact with the nutrient medium. In other aspects of this embodiment, a three-dimensional matrix may be partially immersed in the nutrient medium so that, e.g., about 50% to about 100%, about 70% to about 100%, about 80% to about 100%, about 90% to about 100%, about 95% to about 100%, or about 99% to about 100% of the three-dimensional matrix may be in contact with the nutrient medium.

Aspects of the present specification provide, in part, assaying a population of cells for differentiation. Differentiation, or cell differentiation, refers to a process where a less specialized cell becomes a more specialized cell type. Differentiation dramatically changes a cell's size, shape, membrane potential, metabolic activity, and responsiveness to signals. These changes are largely controlled through the regulation of gene expression. Cell differentiation is thus a transition of a cell from one cell type to another and it involves a switch from one pattern of gene expression to another and the resulting shift in protein expression and cellular function. As such, biomarkers indicative of a certain cell type can be used as a signature to identify cells that have differentiated into that cell type.

Biomarkers indicative of cell differentiation can be detected using a wide range of assays. A biomarker can refer to the presence or absence of a cellular component like a polynucleotide or polypeptide and/or the presence of absence of a cellular activity like an enzymatic activity or a secretion activity. Non-limiting examples include polynucleotide-based detection assays, polypeptide-based detection assays, activity-based assays, and morphology-based assays.

Polynucleotide-based detection assays detect a biomarker by monitoring gene expression and the production of RNA using, e.g., PCR-based assays like RT-PCR, probe-hybridization assays like northern blotting, or particle analyzer-based assays. Polypeptide-based detection assays detect a biomarker by monitoring protein synthesis and the production of polypeptides using, e.g., immunoblotting-based assays like ELISA or western blotting, or particle analyzer-based assays. Activity-based assays detect a biomarker by monitoring a cell function using, e.g., an enzyme activity assay, a secretion assay, a membrane potential assay, a cell-death assay like an apoptotic assay or a necrosis assay. Morphology-based assays detect a biomarker by monitoring changes is cellular appearance using, e.g., microscopy. Such assays are known to a person of skill in the art.

### EXAMPLES

The following examples illustrate representative embodiments now contemplated, but should not be construed to limit the disclosed methods.

### Example 1

To make a crosslinked hyaluronic acid/collagen gel matrix, lyophilized hyaluronic acid polymers, 2 MDa molecular weight, (HTL Biotech) were dissolved in a concentrated human collagen(I) solution in 0.01 N hydrochloric acid and sodium chloride was then added at 0.9% (w/w). 2-(morpholino)ethanesulfonic acid was added at 100 mM to the solution, allowed to react for 1 hour, and the solution then homogenized by syringe-to-syringe mixing. The pH of the solution was adjusted to 5.4 by addition of 1 N sodium hydroxide. 1-Ethyl-3-(3-dimethylaminopropyl)carbodiimide (50 mM) and N-hydroxysulfosuccinimide sodium salt (50 mM) were added to the hyaluronic acid/collagen solution and quickly mixed. The solution was transferred to a glass vial and centrifuged for 5 minutes at 4000 RPM to remove air bubbles. The resulting matrix was allowed to react for 16 hours at 4° C. The matrix was then particulated through a 100 micron pore-sized mesh. Following sizing, the gel was purified by dialysis through a 20 kDa molecular-weight cut-off cellulose ester membrane against 70% isopropanol for 3 hours at 4° C. Dialysis was then continued against sterile phosphate buffer, pH 7.4, for 48 hours at 4° C. with four changes of buffer. The matrix was then dispensed into syringes under aseptic conditions.

This procedure was used to produce gel matrices with varying concentrations of hyaluronic acid and collagen. When required, human collagen(I) in 0.01 N hydrochloric acid was concentrated from 3 mg/mL to the desired reaction concentration in 20 kDa molecular-weight cut-off centrifugal filtration devices. A 50 mL sample of each matrix was synthesized, sterilized by exposure to 70% isopropanol, and purified by dialysis against phosphate buffer, pH 7.4. The matrices produced are described in Table 1.

TABLE 1
Hyaluronic acid-human collagen(I)
gel matrices

| | [HA] | [Col(I)] |
|---|---|---|
| Sample ID | (mg/mL) | (mg/mL) |
| A | 3 | 3 |
| B | 12 | 6 |
| C | 16 | 8 |
| D | 12 | 12 |
| E | 24 | 12 |

### Example 2

To test for the ability of a three-dimensional matrix to support cell differentiation, human adipose-derived stem cells were cultured in matrices comprising matrix polymers and subsequently assayed for biomarkers indicative of adipocyte differentiation. About 1×10⁶ hASC cells were encapsulated in 50 μL of matrix comprising either 12 mg/mL of hyaluronic acid and 6 mg/mL collagen type 1 polymers or 12 mg/mL of hyaluronic acid polymers alone. Once seeded, the three-dimensional matrices were placed in a 0.4 μm transwell in a manner that allowed cell culture media both above and below the transwell insert. The cell culture media was supplemented with factors known to support stem cell differentiation into adipocytes (StemPro Adipogenesis Differentiation Kit; Cat#A10070-01, Invitrogen Corp., Carlsbad, Calif.). The matrices were cultured at 37° C. with 5% CO₂ in a tissue culture incubator over a period of 35 days. Leptin and adiponectin levels present in the media were measured by enzyme-linked immunosorbent assay (ELISA) at 7, 14, 21, 28, and 35 days. All results pertaining to the hyaluronic acid-collagen(I) matrices are presented as a relative comparison to the hyaluronic acid (without collagen) matrices.

The results indicate that hASCs cultured in HA-collagen(I) gels [Sample ID HA-CN (12.6)] released increasing levels of leptin and adiponectin with extended time in culture (Table 2). HA-collagen(I) cultures also produced significantly higher levels of leptin (at the 21, 28, and 35 day time points) than the HA gels without collagen(I) [Sample ID HA without CN] cultures (Table 2). Two-sample t-tests resulted in p-values of 0.008, 0.002, and 0.003 for leptin levels from the HA-CN (12.6) and HA without CN cultures at 21, 28, and 35 days respectively. Adiponectin levels at the 28 and 35 day time points were significantly different in the HA-CN and HA gels without CN cultures (p-values from two sample t-tests were 0.026 and 0.010 respectively).

TABLE 2
Secretion of leptin and adiponectin in
hyaluronic acid-collagen(I) matrices

| | Leptin Levels (ng/mL) | | Adiponectin Levels (ng/mL) | |
|---|---|---|---|---|
| Time | HA-CN (12.6) | HA without CN | HA-CN (12.6) | HA without CN |
| Day 7 | 0.154 ± 0.019 | 0.085 ± 0.013 | 1.073 ± 0.220 | −0.061 ± 0.357 |
| Day 14 | 0.221 ± 0.044 | 0.093 ± 0.041 | 0.283 ± 0.381 | 0.549 ± 0.811 |
| Day 21 | 0.619 ± 0.077 | 0.237 ± 0.068 | 2.914 ± 1.451 | 2.011 ± 1.403 |
| Day 28 | 0.749 ± 0.064 | 0.282 ± 0.043 | 7.054 ± 1.235 | 3.643 ± 0.721 |
| Day 35 | 0.588 ± 0.040 | 0.192 ± 0.064 | 18.940 ± 2.360 | 3.874 ± 1.086 |

Test matrices were also examined for evidence of adipocyte morphology using fluorescent microscopy. At the end of 35 days, matrices were fixed with 4% paraformaldehyde in sodium cacodylate. The fixed matrices were stained with a fluorescent dye used to visualize the intracellular lipid droplets found in adipocytes (AdipoRed Assay Reagent; Cat#PT-7009, Lonza) and with a nuclear dye to confirm the presence of cell nuclei (DAPI; Cat#D3571, Invitrogen Corp., Carlsbad, Calif.). Stained matrices were then examined using fluorescence microscopy. Microscopic examination revealed adipocyte morphology as shown by positive fluorescent dye staining of lipid droplets that co-localize with nuclei of cells (FIG. 2). These results indicate that matrices support morphological differentiation of hASCs into adipocytes and confirm the ELISA data that hASCs are differentiating into mature adipocytes.

The results obtained from the three-dimensional assay to evaluate adipogenic differentiation demonstrate that hyaluronic acid-collagen(I) matrices can improve fat grafting outcomes and other potential tissue engineering-based approaches to adipose tissue repair or regeneration by showing that hyaluronic acid-collagen(I) matrices stimulate the differentiation of human adipose-derived stem cells into mature adipocytes.

In closing, it is to be understood that although aspects of the present specification have been described with reference to the various embodiments, one skilled in the art will readily appreciate that the specific examples disclosed are only illustrative of the principles of the subject matter disclosed in the present specification. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

All patents, patent publications, and other publications referenced and identified in the present specification are individually and expressly incorporated herein by reference in their entirety for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A three-dimensional in vitro cell-based method to assess a test matrix polymer ability to support differentiation of a population of cells, the method comprising the step of:
a) culturing a three-dimensional matrix comprising the test matrix polymer and the population of cells in a nutrient medium using an apparatus, wherein the three-dimensional matrix comprises a top surface and a bottom surface, and wherein the apparatus is configured to allow contact of the nutrient medium on the top surface and the bottom surface of the three-dimensional matrix;
b) assaying the population of cells for differentiation, wherein detection of differentiation is indicative that the test matrix polymer stimulates differentiation of a population of cells.

2. The method according to claim 1, wherein the population of cells comprises stem cells.

3. The method according to claim 2, wherein the stem cells comprise embryonic stem cells or adult stem cells.

4. The method according to claim 2, wherein the stem cells comprise totipotent stem cells, pluripotent stem cells, multipotent stem cells, oligopotent stem cells, or unipotent stem cells.

5. The method according to claim 2, wherein the stem cells comprise lineage-restricted stem cells.

6. The method according to claim 2, wherein the stem cells comprise mesenchymal stem cells, adipose-derived stem cells, endothelial stem cells, or dental pulp stem cells.

7. The method according to claim 1, wherein the test matrix polymer comprises a polysaccharide, a polypeptide, a polyester, or any combination thereof.

8. The method according to claim 7, wherein the polysaccharide is a cellulose, an agarose, a chitosan, a chitin, a glycosaminoglycan, or a derivative thereof.

9. The method according to claim 8, wherein the glycosaminoglycan is chondroitin sulfate, dermatan sulfate, keratan sulfate, hyaluronan, or a derivative thereof.

10. The method according to claim 7, wherein the polypeptide is an elastic protein or a derivative thereof.

11. The method according to claim 10, wherein the elastic protein is a silk protein, a resilin, a resilin-like polypeptides (RLPs), an elastin, an elastin-like polypeptides (ELPs), a silk protein-elastin-like polypeptides (SELPs), a gluten, an abductin, a byssus, a keratin, a gelatin, a lubricin, a collagen or a derivative thereof.

12. The method according to claim 7, wherein the polyester is D-lactic acid, L-lactic acid, racemic lactic acid, glycolic acid, caprolactone, or a derivative thereof.

13. The method according to claim 7, wherein the biomaterial comprises a macromolecular matrix comprising a hyaluronic acid cross-linked to a collagen.

14. The method according to claim 1, wherein the biomaterial comprises a hydrogel.

15. The method according to claim 1, wherein the three-dimensional matrix is completely submerged in the medium.

16. The method according to claim 1, wherein the apparatus comprises a permeable support.

17. The method according to claim 16, wherein the three-dimensional matrix is suspended in the nutrient medium on the permeable support.

18. The method according to claim 16, wherein the permeable support comprises pores having a diameter of about 0.1 μm to about 30 μm.

19. The method according to claim 16, wherein the permeable support has a thickness of about 1 μm to about 100 μm.

20. The method according to claim 1, wherein the three-dimensional matrix further comprises a test compound.

21. A method of screening a material for its ability to stimulate cell growth and/or differentiation, the method comprising:
incubating cells in a three-dimensional culture to determine growth and/or differentiation of the cells;
wherein the three-dimensional culture comprises the cells dispersed in a three-dimensional matrix comprising a test material; and
wherein the three-dimensional matrix is in contact with a nutrient medium such that a nutrient in the nutrient medium contacts from opposite sides the three-dimensional matrix of the biomaterial.
